(19) United States
(12) Patent Application Publication
(10) Pub. No.: US 2020/0332017 A1
(43) Pub. Date: Oct. 22, 2020

(54) ANTIBODIES OR ANTIGEN-BINDING PORTIONS BINDING CD40 AND USES THEREOF

(71) Applicant: Salubris (Chengdu) Biotech Co., Ltd., Chengdu (CN)

(72) Inventors: John Li, Reston, VA (US); Shengwei Li, Chengdu, Sichuan (CN); Yang Xu, Changsha, Hunan (CN); Ming Zhou, Dezhou, Shandong (CN); Mingjiu Chen, Nanjing, Jiangsu (CN); Shukai Xia, Nanjing, Jiangsu (CN)

(21) Appl. No.: 16/859,299

(22) Filed: Apr. 27, 2020

Related U.S. Application Data
(63) Continuation of application No. PCT/CN2020/080295, filed on Mar. 20, 2020.

(30) Foreign Application Priority Data
Mar. 20, 2020 (CN) 202010198584.3

(51) Int. Cl. C07K 16/28

(52) U.S. Cl. CPC C07K 16/2878; C07K 2317/565; A61K 2039/505; C07K 2317/75; C07K 2317/24; C07K 2317/76

(57) ABSTRACT

The present invention provides an isolated monoclonal antibody, or an antigen-binding portion thereof, specifically binding to CD40. The present invention further provides a pharmaceutical composition comprising the antibody, or the antigen-binding portion thereof, as well as a treatment method using the antibody or the antigen-binding portion thereof, or the pharmaceutical composition.

### FIELD OF THE INVENTION

The present invention relates generally to an isolated monoclonal antibody, particularly a mouse, chimeric or humanized monoclonal antibody, or the antigen-binding portion thereof, that specifically binds to human CD40 with high affinity and functionality. A nucleic acid molecule encoding the antibody or the antigen-binding portion thereof, an expression vector, a host cell and a method for expressing the antibody are also provided. The present invention further provides an immunoconjugate, a bispecific molecule, an oncolytic virus, and a pharmaceutical composition comprising the antibody or the antigen-binding portion thereof, as well as a diagnostic and treatment method using an anti-CD40 antibody or the antigen-binding portion of the invention.

### BACKGROUND OF THE INVENTION

CD40, a member of the TNF receptor superfamily, is expressed on antigen presenting cells such as B cells, macrophages, and dendritic cells as a transmembrane costimulatory protein. It binds to CD40L, its major ligand expressed on activated T cells under inflammatory conditions (Younes et al., (1998) Br. J. Haematol 100:135-141; Grewal et al., (1998) Annu. Rev. Immunol. 16:111-135). CD40-CD40L interaction plays an important role in activating innate and adaptive immunity. The engagement of CD40 by CD40L results in CD40 multimerization, generation of activation signals for antigen presenting cells, growth and proliferation of immune cells, and production of cytokines and chemokines (Ara A et al., (2018) Immunotargets Ther 7: 55-61).

CD40 is also expressed on non-immune cells and tumors (Costello et al., (1999) Immunol Today 20(11): 488-493; Tong et al., (2003) Cancer Gene Ther 10(1): 1-13; Lee et al., (2014) Curr Cancer Drug Targets 14(7): 610-620; Ara A et al., (2018) supra), and has been reported to be involved in pathologies of several inflammatory diseases, including autoimmune diseases, atherothrombosis, cancers, and respiratory diseases. Especially, CD40 expression has also been found in many human cancers, including B-cell malignancies, lung, bladder, gastric, breast and ovarian cancers (Lee et al., (1999) Proc Natl Acad Sci USA 96:9136-9141; Stamenkovic et al., (1989) EMBO J. 8:1403-1410).

The interaction of CD40 with CD40L results in B cell activation and proliferation of normal B cells. However CD40-mediated signaling in B cell-derived tumor lines can cause activation-induced cell death. The strength of the activation signal is important for activation-induced tumor cell death (Grafton et al., (1997) Cell. Immunol. 182:45-56).

Depending on the action mechanisms, there are generally two types of anti-CD40 antibodies, agonistic ones that activate or induce CD40 signaling upon binding CD40, and antagonistic ones that block or inhibit CD40 signaling that may be induced by CD40L engagement. CP-870,893 or Selicrelumab (Pfizer and VLST) is an agonistic anti-CD40 antibody, and has shown clinical efficacy in a number of settings of patients with advanced cancers. For example, objective tumor responses were reported in melanoma and pancreatic carcinoma (about 20%) (Vonderheide et al., (2013) Clin Cancer Res. 19(5):1035-1043). Dacetuzumab (Settte Geneticscs), a weaker CD40 agonist than CP-870,893, has anti-tumor activity in diffuse large B cell lymphoma (also referred to as DLBCL), multiple myeloma and CLL, and has been tested in combination with Rituximab and Gemcitabine in treatment of relapsed or refractory DLBCL (Advani R et al., (2009) J Clin Oncol. 27:4371-4377; Furman R R et al., (2010) Leuk Lymphoma. 51:228-235; Forero-Torres A et al., (2012) Leuk Lymphoma 54(2):277-283). As an antagonistic anti-CD40 antibody, Lucatumumab (Novatris) has been tested in clinical trials for treating multiple myeloma and chronic lymphocytic leukemia (Hassan S B et al., (2014) Immunopharmacol immunotoxicol 36(2):96-104).

There remains a need for more CD40 antibodies with improved pharmaceutical characteristics.

### SUMMARY OF THE INVENTION

The present invention provides an isolated monoclonal antibody, for example, a mouse, human, chimeric or humanized monoclonal antibody, or an antigen-binding portion, that binds to CD40 (e.g., the human CD40, and monkey CD40) and has good binding affinity to CD40, and high blocking activity on the binding of CD40 to CD40L.

The antibody or antigen-binding portion of the invention can be used for a variety of applications, including detection of the CD40 protein, and treatment and prevention of CD40 associated diseases, such as autoimmune diseases, atherothrombosis, cancers, and respiratory diseases.

Accordingly, in one aspect, the invention pertains to an isolated monoclonal antibody (e.g., a mouse, chimeric or humanized antibody), or an antigen-binding portion thereof, that binds CD40, having a heavy chain variable region that comprises a CDR1 region, a CDR2 region and a CDR3 region, wherein the CDR1 region, the CDR2 region and the CDR3 region comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 1, 2 and 3, respectively; or (2) SEQ ID NOs: 37, 38 and 3, respectively; or (3) SEQ ID NOs: 39, 40 and 41, respectively; or (4) SEQ ID NOs: 42, 43 and 3, respectively; or (5) SEQ ID NOs: 44, 45 and 46, respectively; or (6) SEQ ID NOs: 4, 5 and 6, respectively; or (7) SEQ ID NOs: 47, 48 and 6, respectively; or (8) SEQ ID NOs: 49, 50 and 51, respectively; or (9) SEQ ID NOs: 52, 53 and 6, respectively; or (10) SEQ ID NOs: 54, 55 and 56, respectively.

In one aspect, an isolated monoclonal antibody, or an antigen-binding portion thereof, of the present invention comprises a heavy chain variable region comprising an amino acid sequence having at least 85%, 86%, 87%, 88%, 89%, 90%, 91%, 92%, 93%, 94%, 95%, 96%, 97%, 98%, 99% or 100% identity to SEQ ID NOs: 13, 14 (X1=C), 15 (X1=F), or 16, wherein the antibody or antigen-binding fragment thereof binds to CD40.

In one aspect, an isolated monoclonal antibody, or an antigen-binding portion thereof, of the present invention comprises a heavy chain constant region having an amino acid sequence at least 85%, 86%, 87%, 88%, 89%, 90%, 91%, 92%, 93%, 94%, 95%, 96%, 97%, 98%, 99% or 100% identity to SEQ ID NO: 24.

In one aspect, an isolated monoclonal antibody, or an antigen-binding portion thereof, of the present invention comprises the heavy chain comprises an amino acid sequence having at least 85%, 86%, 87%, 88%, 89%, 90%, 91%, 92%, 93%, 94%, 95%, 96%, 97%, 98%, 99% or 100% identity to (1) SEQ ID NO: 26; or (2) SEQ ID NO: 27 (X1=C); or (3) SEQ ID NO: 28 (X1=F); or (4) SEQ ID NO: 29, respectively.

In one aspect, an isolated monoclonal antibody, or an antigen-binding portion thereof, of the present invention comprises a light chain variable region that comprises a CDR1 region, a CDR2 region and a CDR3 region, wherein the CDR1 region, the CDR2 region, and the CDR3 region comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 7, 8 and 9, respectively; or (2) SEQ ID NOs: 57, 58 and 9, respectively; or (3) SEQ ID NOs: 59, 60 and 61, respectively; or (4) SEQ ID NOs: 10, 11 and 12, respectively; or (5) SEQ ID NOs: 62, 63 and 12, respectively; or (6) SEQ ID NOs: 64, 65 and 66, respectively.

In one aspect, an isolated monoclonal antibody, or an antigen-binding portion thereof, of the present invention comprises a light chain variable region comprising an amino acid sequence having at least 85%, 86%, 87%, 88%, 89%, 90%, 91%, 92%, 93%, 94%, 95%, 96%, 97%, 98%, 99% or 100% identity to SEQ ID NOs: 17, 18 (X1=S, X2=N, X3=F), 19 (X1=A, X2=N, X3=F), 20 (X1=S, X2=Y, X3=F), 21 (X1=S, X2=N, X3=Y), 22 (X1=A, X2=Y, X3=Y), or 23 (X1=M, X2=N, X3=A), wherein the antibody or antigen-binding fragment thereof binds to CD40.

In one aspect, an isolated monoclonal antibody, or an antigen-binding portion thereof, of the present invention comprises a light chain constant region having an amino acid sequence at least 85%, 86%, 87%, 88%, 89%, 90%, 91%, 92%, 93%, 94%, 95%, 96%, 97%, 98%, 99% or 100% identity to SEQ ID NO: 25, linked to the light chain variable region.

In one aspect, an antibody, or the antigen-binding portion thereof, of the present invention comprises the light chain comprises having an amino acid sequence at least 85%, 86%, 87%, 88%, 89%, 90%, 91%, 92%, 93%, 94%, 95%, 96%, 97%, 98%, 99% or 100% identity to (1) SEQ ID NO: 30; or (2) SEQ ID NO: 31 (X1=S, X2=N, X3=F); or (3) SEQ ID NO: 32 (X1=A, X2=N, X3=F); or (4) SEQ ID NO: 33 (X1=S, X2=Y, X3=F); or (5) SEQ ID NO: 34 (X1=S, X2=N, X3=Y); or (6) SEQ ID NO: 35 (X1=A, X2=Y, X3=Y); or (7) SEQ ID NO: 36 (X1=M, X2=N, X3=A), respectively.

In one aspect, an isolated monoclonal antibody, or an antigen-binding portion thereof, of the present invention comprises a heavy chain variable region and a light chain variable region each comprising a CDR1 region, a CDR2 region and a CDR3 region, wherein the heavy chain variable region CDR1, CDR2 and CDR3, and the light chain variable region CDR1, CDR2 and CDR3 comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 1, 2, 3, 7, 8 and 9, respectively; or (2) SEQ ID NOs: 37, 38, 3, 7, 8, and 9, respectively; or (3) SEQ ID NOs: 39, 40, 41, 57, 58, and 9, respectively; or (4) SEQ ID NOs: 42, 43, 3, 7, 8, and 9, respectively; or (5) SEQ ID NOs: 44, 45, 46, 59, 60, and 61, respectively; or (6) SEQ ID NOs: 4, 5, 6, 10, 11, and 12, respectively; or (7) SEQ ID NOs: 47, 48, 6, 10, 11, and 12, respectively; or (8) SEQ ID NOs: 49, 50, 51, 62, 63, and 12, respectively; or (9) SEQ ID NOs: 52, 53, 6, 10, 11, and 12, respectively; or (10) SEQ ID NOs: 54, 55, 56, 64, 65, and 66, respectively, wherein the antibody or antigen-binding fragment thereof binds to CD40.

In one embodiment, an isolated monoclonal antibody, or the antigen-binding portion thereof, of the present invention comprises a heavy chain variable region and a light chain variable region, the heavy chain variable region and the light chain variable region comprising amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 13 and 17, respectively; or (2) SEQ ID NOs: 14 (X1=C) and 18 (X1=S, X2=N, X3=F), respectively; or (3) SEQ ID NOs: 14 (X1=C) and 19 (X1=A, X2=N, X3=F), respectively; or (4) SEQ ID NOs: 14 (X1=C) and 20 (X1=S, X2=Y, X3=F), respectively; or (5) SEQ ID NOs: 14 (X1=C) and 21 (X1=S, X2=N, X3=Y), respectively; or (6) SEQ ID NOs: 14 (X1=C) and 22 (X1=A, X2=Y, X3=Y), respectively; or (7) SEQ ID NOs: 15 (X1=F) and 18 (X1=S, X2=N, X3=F), respectively; or (8) SEQ ID NOs: 15 (X1=F) and 22 (X1=A, X2=Y, X3=Y), respectively; or (9) SEQ ID NOs: 16 and 23 (X1=M, X2=N, X3=A), respectively, wherein the antibody or antigen-binding fragment thereof binds to CD40. The amino acids set forth in SEQ ID NOs.: 13, 16, 17, 23 (X1=M, X2=N, X3=A), 24 and 25 can be encoded by SEQ ID NOs.: 68-73, respectively.

In one embodiment, an isolated monoclonal antibody, or the antigen-binding portion thereof, of the present invention comprises a heavy chain and a light chain, the heavy chain comprising a heavy chain variable region and a heavy chain constant region, the light chain comprising a light chain variable region and a light chain constant region, wherein, the heavy chain constant region comprises an amino acid sequence having at least 85%, 86%, 87%, 88%, 89%, 90%, 91%, 92%, 93%, 94%, 95%, 96%, 97%, 98%, 99% or 100% identity SEQ ID No: 24, and the light chain constant region comprises an amino acid sequences having at least 85%, 86%, 87%, 88%, 89%, 90%, 91%, 92%, 93%, 94%, 95%, 96%, 97%, 98%, 99% or 100% identity SEQ ID No: 25, and the heavy chain variable region and the light chain variable region comprise amino acid sequences described above, wherein the antibody or antigen-binding fragment thereof binds to CD40.

In one embodiment, an isolated monoclonal antibody, or the antigen-binding portion thereof, of the present invention comprises a heavy chain and a light chain comprising amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 26 and 30, respectively; or (2) SEQ ID NOs: 27 (X1=C) and 31 (X1=S, X2=N, X3=F), respectively; or (3) SEQ ID NOs: 27 (X1=C) and 32 (X1=A, X2=N, X3=F), respectively; or (4) SEQ ID NOs: 27 (X1=C) and 33 (X1=S, X2=Y, X3=F), respectively; or (5) SEQ ID NOs: 27 (X1=C) and 34 (X1=S, X2=N, X3=Y), respectively; or (6) SEQ ID NOs: 27 (X1=C) and 35 (X1=A, X2=Y, X3=Y), respectively; or (7) SEQ ID NOs: 28 (X1=F) and 31 (X1=S, X2=N, X3=F), respectively; or (8) SEQ ID NOs: 28 (X1=F) and 35 (X1=A, X2=Y, X3=Y), respectively; or (9) SEQ ID NOs: 29 and 36 (X1=M, X2=N, X3=A), respectively, wherein the antibody or antigen-binding fragment thereof binds to CD40.

The antibody, or the antigen-binding portion thereof, of the present invention (a) binds human or monkey CD40; (b) blocks human CD40-human CD40L interaction; and (c) activates CD40 signaling. In one embodiment, the antibody of the present invention, is a mouse, human, chimeric or humanized antibody. The antibody of the present invention in some embodiments comprises or consists of two heavy chains and two light chains, wherein each heavy chain comprises the heavy chain constant region, heavy chain variable region or CDR sequences mentioned above, and each light chain comprises the light chain constant region, light chain variable region or CDR sequences mentioned above, wherein the antibody binds to CD40. The antibody of the invention can be a full-length antibody, for example, of an IgG1, IgG2 or IgG4 isotype. The light chain constant region may be a kappa or lambda constant region. The antibody of the present invention in other embodiments may be a single chain variable fragment (scFv) antibody, or antibody fragments, such as Fab or F(ab′)₂ fragments.

The antibody, or antigen-binding portion thereof, of the present invention has comparable or higher binding affinity to human CD40 and monkey CD40 than prior art anti-CD40 antibodies such as Dacetuzumab and Selicrelumab, inhibits the binding of CD40L to CD40, and activates CD40 signaling.

Nucleic acid molecules encoding the antibodies, or antigen-binding portions thereof, of the invention are also encompassed by the invention, as well as expression vectors comprising such nucleic acids and host cells comprising such expression vectors. A method for preparing an anti-CD40 antibody or an antigen-binding portion thereof using the host cell comprising the expression vector is also provided, comprising steps of (i) expressing the antibody in the host cell and (ii) isolating the antibody from the host cell or its cell culture.

The invention also provides an immunoconjugate comprising an antibody of the invention, or antigen-binding portion thereof, linked to a therapeutic agent, such as a cytotoxin. The invention also provides a bispecific molecule comprising an antibody, or antigen-binding portion thereof, of the invention, linked to a second functional moiety (e.g., a second antibody) having a different binding specificity than said antibody, or antigen-binding portion thereof. I The antibody or an antigen binding portion thereof of the present invention can also be encoded by or used in conjunction with an oncolytic virus.

Pharmaceutical compositions comprising the antibody, or the antigen-binding portion thereof, or the immunoconjugate, the bispecific molecule, the oncolytic virus, of the invention, and a pharmaceutically acceptable carrier, are also provided.

In yet another aspect, the invention provides a method of modulating an immune response in a subject comprising administering to the subject the antibody, or antigen-binding portion thereof, of the invention such that the immune response in the subject is modulated. Preferably, the antibody, or the antigen-binding portion thereof, of the invention enhances, stimulates or increases the immune response in the subject. Preferably, a method for treating a cancer disease in a subject, comprises administering to the subject a therapeutically effective amount of the isolated monoclonal antibody, or the antigen-binding portion thereof, of the present invention, or the pharmaceutical composition thereof. The method further comprises administering an immune checkpoint antibody, a costimulatory antibody, a chemotherapeutic agent, and/or a cytokine.

In another aspect, the invention provides a method for treating inflammatory diseases, or infectious diseases in a subject in need thereof, comprising administering to the subject a therapeutically effective amount of the antibody, or antigen-binding portion thereof, of the invention. In some embodiments, the method comprises administering a composition, a bispecific molecule, an immunoconjugate, or an antibody-encoding or antibody-bearing oncolytic virus of the invention. In some embodiments, additional agents can be administered with the antibody, or an antigen-binding portion thereof, of the invention, such as anti-inflammatory agents and/or antimicrobial or antiviral agents. In some embodiments, the inflammatory diseases include autoimmune diseases, atherothrombosis, and respiratory diseases.

In a further aspect, the invention provides a method of inhibiting tumor growth in a subject, comprising administering to a subject a therapeutically effective amount of the antibody, or antigen-binding portion thereof, of the present invention. The tumor may be a solid or non-solid tumor, including, but not limited to, B cell lymphoma, chronic lymphocytic leukemia, multiple myeloma, melanoma, colon adenocarcinoma, pancreas cancer, colon cancer, gastric intestine cancer, prostate cancer, bladder cancer, kidney cancer, ovary cancer, cervix cancer, breast cancer, lung cancer, and nasopharynx cancer. In some embodiments, the method comprises administering a composition, a bispecific molecule, an immunoconjugate, or an antibody-encoding or antibody-bearing oncolytic virus of the invention, or alternatively a nucleic acid molecule capable of expressing the same in the subject. In some embodiments, at least one additional anti-cancer antibody can be administered with the antibody, or an antigen-binding portion thereof, of the invention, such as an anti-VISTA antibody, an anti-PD-1 antibody, an anti-PD-L1 antibody, an anti-LAG-3 antibody, an anti-CTLA-4 antibody, an anti-TIM 3 antibody, an anti-STATS antibody, and/or an anti-ROR1 antibody. In yet another embodiment, an antibody, or an antigen-binding portion thereof, of the invention is administered with a cytokine (e.g., IL-2, IL-21, GM-CSF and/or IL-4), or a costimulatory antibody (e.g., an anti-CD137 and/or anti-GITR antibody). In another embodiment, an antibody, or an antigen-binding portion thereof, of the invention is administered with a chemotherapeutic agent, which may be a cytotoxic agent, such as epirubicin, oxaliplatin, and/or 5-fluorouracil (5-FU). The antibodies of the present invention can be, for example, mouse, human, chimeric or humanized antibodies.

Other features and advantages of the instant invention will be apparent from the following detailed description and examples, which should not be construed as limiting. The contents of all references, Genbank entries, patents and published patent applications cited throughout this application are expressly incorporated herein by reference.

### BRIEF DESCRIPTION OF THE DRAWINGS

FIG. 1 shows the binding capacities of mouse antibodies C1C1 and 1B2 to human CD40.

FIG. 2 shows the binding capacities of the antibodies to 293T cells expressing human CD40, wherein FIG. 2A and FIG. 2B show the binding capacities of mouse antibodies C1C1 (FIG. 2A) and 1B2 (FIG. 2B) to 293T cells expressing human CD40.

FIG. 3 shows the blocking abilities of mouse antibodies C1C1 and 1B2 on the binding of human CD40 to human CD40L.

FIG. 4 shows the abilities of the antibodies to block the binding of Benchmark Selicrelumab to human CD40, wherein FIG. 4A and FIG. 4B show the abilities of mouse antibodies C1C1 (FIG. 4A) and 1B2 (FIG. 4B) to block the binding of Benchmark Selicrelumab to human CD40.

FIG. 5 shows the agonistic activities of the antibodies, wherein FIG. 5A-5B show the agonistic activities of mouse antibodies C1C1 (5A) and 1B2 (5B).

FIG. 6 shows the binding capacities of mouse C1C1, chimeric C1C1 and humanized C1C1-V1 to humanized C1C1-V5 antibodies to human CD40.

FIG. 7 shows the binding capacity of chimeric C1C1 and humanized antibody huC1C1-V7 to human CD40.

FIG. 8 shows the binding capacities of mouse C1C1, chimeric C1C1 and humanized C1C1-V7 antibodies to 293T cells expressing human CD40.

FIG. 9 shows the blocking abilities of mouse C1C1, chimeric C1C1 and humanized C1C1-V7 antibodies on the binding of human CD40 to human CD40L.

FIG. 10 shows the abilities of mouse C1C1, chimeric C1C1 and humanized C1C1-V7 antibodies to block the binding of Benchmark Selicrelumab to human CD40.

FIG. 11 shows the agonistic activities of mouse C1C1, chimeric C1C1 and humanized C1C1-V7 antibodies.

### DETAILED DESCRIPTION OF THE INVENTION

To ensure that the present disclosure may be more readily understood, certain terms are first defined. Additional definitions are set forth throughout the detailed description.

The term “CD40” refers to tumor necrosis factor receptor superfamily member 5. The term “CD40” comprises variants, isoforms, homologs, orthologs and paralogs. For example, an antibody specific for a human CD40 protein may, in certain cases, cross-react with a CD40 protein from a species other than human, such as monkey. In other embodiments, an antibody specific for a human CD40 protein may be completely specific for the human CD40 protein and exhibit no cross-reactivity to other species or of other types, or may cross-react with CD40 from certain other species but not all other species.

The term “human CD40” refers to a CD40 protein having an amino acid sequence from a human, such as the amino acid sequence of human CD40 having a Genbank accession number of NP_001241.1. The terms “monkey or rhesus CD40” and “mouse CD40” refer to monkey and mouse CD40 sequences, respectively, e.g. those with the amino acid sequences having Genbank Accession Nos. NP_001252791.1 and NP_035741.2, respectively.

The term “immune response” refers to a biological response within a vertebrate against foreign agents, which response protects the organism against these agents and diseases caused by them. An immune response is mediated by the action of a cell of the immune system (for example, lymphocytes, natural killer cell, antigen presenting cells, or phagocytic cells) and soluble macromolecules (including antibodies, cytokines, and complement) produced by any of these cells or the liver that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues.

The term “antibody” as referred to herein includes whole antibodies and any antigen binding fragment (i.e., “antigen-binding portion”) or single chains thereof. Whole antibodies are glycoproteins comprising two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g., a CD40 protein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR); and (viii) a nanobody, a heavy chain variable region containing a single variable domain and two constant domains. Furthermore, although the two domains of the Fv fragment, V_{L} and V_{H}, are coded by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., (1988) Science 242:423-426; and Huston et al., (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds a CD40 protein is substantially free of antibodies that specifically bind antigens other than CD40 proteins). An isolated antibody that specifically binds a human CD40 protein may, however, have cross-reactivity to other antigens, such as CD40 proteins from other species. Moreover, an isolated antibody can be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “mouse antibody”, as used herein, is intended to include antibodies having variable regions in which both the framework and CDR regions are derived from mouse germline immunoglobulin sequences. Furthermore, if the antibody contains a constant region, the constant region also is derived from mouse germline immunoglobulin sequences. The mouse antibodies of the invention can include amino acid residues not encoded by mouse germline immunoglobulin sequences (e.g., mutations introduced by random or site-specific mutagenesis in vitro or by somatic mutation in vivo). However, the term “mouse antibody”, as used herein, is not intended to include antibodies in which CDR sequences derived from the germline of another mammalian species have been grafted onto mouse framework sequences.

The term “chimeric antibody” refers to an antibody made by combining genetic material from a nonhuman source with genetic material from a human being. Or more generally, a chimeric antibody is an antibody having genetic material from a certain species with genetic material from another species.

The term “humanized antibody”, as used herein, refers to an antibody from non-human species whose protein sequences have been modified to increase similarity to antibody variants produced naturally in humans.

The term “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

As used herein, an antibody that “specifically binds to human CD40” is intended to refer to an antibody that binds to human CD40 protein (and possibly a CD40 protein from one or more non-human species) but does not substantially bind to non-CD40 proteins. Preferably, the antibody binds to human CD40 protein with “high affinity”, namely with a K_{D} of 5.0×10⁻⁸ M or less, more preferably 1.0×10⁻⁸ M or less, and more preferably 7.0×10⁻⁹ M or less.

The term “does not substantially bind” to a protein or cells, as used herein, means does not bind or does not bind with a high affinity to the protein or cells, i.e. binds to the protein or cells with a K_{D} of 1.0×10⁻⁶ M or more, more preferably 1.0×10⁻⁵ M or more, more preferably 1.0×10⁻⁴ M or more, more preferably 1.0×10⁻³ M or more, even more preferably 1.0×10⁻² M or more.

The term “high affinity” for an IgG antibody refers to an antibody having a K_{D} of 1.0×10⁻⁶ M or less, more preferably 5.0×10⁻⁸ M or less, even more preferably 1.0×10⁻⁸ M or less, even more preferably 7.0×10⁻⁹ M or less and even more preferably 1.0×10⁻⁹ M or less for a target antigen. However, “high affinity” binding can vary for other antibody isotypes. For example, “high affinity” binding for an IgM isotype refers to an antibody having a K_{D} of 10⁻⁶ M or less, more preferably 10⁻⁷ M or less, even more preferably 10⁻⁸ M or less.

The term “K_{assoc}” or “Kₐ”, as used herein, is intended to refer to the association rate of a particular antibody-antigen interaction, whereas the term “K_{dis}” or “K_{d}”, as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The term “EC₅₀”, also known as half maximal effective concentration, refers to the concentration of an antibody which induces a response halfway between the baseline and maximum after a specified exposure time.

The term “IC₅₀”, also known as half maximal inhibitory concentration, refers to the concentration of an antibody which inhibits a specific biological or biochemical function by 50% relative to the absence of the antibody.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

The term “therapeutically effective amount” means an amount of the antibody of the present invention sufficient to prevent or ameliorate the symptoms associated with a disease or condition (such as a cancer) and/or lessen the severity of the disease or condition. A therapeutically effective amount is understood to be in context to the condition being treated, where the actual effective amount is readily discerned by those of skill in the art.

The term “agonistic CD40 antibody” or “agonistic anti-CD40 antibody” refers to an anti-CD40 antibody that binds to CD40 and activates or induces CD40 signaling to promote immune cell activation and proliferation as well as cytokine and chemokine production. While the term “antagonistic CD40 antibody” refers to an anti-CD40 antibody that blocks or inhibits CD40 signaling that may be induced by CD40L engagement. The agonistic CD40 antibody may promote a tumor-bearing subject's innate and adaptive immune response to tumors, via elevated antigen presenting ability of APCs, activation of tumor specific CD4+ and CD8+ T cells, secretion of cytokines and chemokines by lymphocytes and monocytes, enhanced tumor cell killings by cytotoxic lymphocytes and NK cells, etc.

Various aspects of the invention are described in further detail in the following subsections.

Anti-CD40 Antibodies Having Increased Binding Affinity to Human CD40 and Better Anti-Tumor Effect

The antibody, or the antigen-binding portion thereof, of the invention specifically binds to human CD40 with comparable, or higher binding affinity as compared to previously described anti-CD40 antibodies, such as Dacetuzumab and Selicrelumab.

Additional functional properties include the capacity to block the binding of CD40 to CD40L, and to activate CD40 signaling.

Preferred antibodies of the invention are humanized monoclonal antibodies. Additionally or alternatively, the antibodies can be, for example, chimeric monoclonal antibodies.

Monoclonal Anti-CD40 Antibody

The preferred antibody of the invention is the monoclonal antibody structurally and chemically characterized as described below and in the following Examples. The amino acid sequence ID numbers of the heavy/light chain variable regions of the antibodies are summarized in Table 1 below, some antibodies sharing the same V_{H} or V_{L}. The heavy chain constant region for the antibodies may be human IgG1 heavy chain constant region having an amino acid sequence set forth in, e.g., SEQ ID NOs: 24, and the light chain constant region for the antibodies may be human kappa constant region having an amino acid sequence set forth in, e.g., SEQ ID NO: 25. These antibodies may also contain mouse IgG1 heavy chain constant region, and/or mouse kappa constant region.

The heavy chain variable region CDRs and the light chain variable region CDRs in Table 1 have been defined by the Kabat numbering system. However, as is well known in the art, CDR regions can also be determined by other systems such as Chothia, and IMGT, AbM, or Contact numbering system/method, based on heavy chain/light chain variable region sequences. The antibody CDRs of the present invention defined by different numbering systems/methods are listed in Table 2.

TABLE 1
Amino acid sequence ID numbers of heavy/light chain variable regions
whose CDRs are defined by the Kabat numbering system

| | SEQ ID NO. | | | | | | | | | | | |
|---|---|---|---|---|---|---|---|---|---|---|---|---|
| | V_{H} | V_{H} | V_{H} | | | | V_{L} | V_{L} | V_{L} | | | |
| Antibody | CDR | CDR | CDR | | | Heavy | CDR | CDR | CDR | | | Light |
| ID | 1 | 2 | 3 | V_{H} | C_{H} | chain | 1 | 2 | 3 | V_{L} | C_{L} | chain |
| Mouse | 1 | 2 | 3 | 13 | 24 | 26 | 7 | 8 | 9 | 17 | 25 | 30 |
| C1C1 | | | | | | | | | | | | |
| huC1C1- | 1 | 2 | 3 | 14, | 24 | 27, | 7 | 8 | 9 | 18, | 25 | 31, |
| V1 | | | | X1 = C | | X1 = C | | | | X1 = S, | | X1 = S, |
| | | | | | | | | | | X2 = N, | | X2 = N, |
| | | | | | | | | | | X3 = F | | X3 = F |
| huC1C1- | 1 | 2 | 3 | 15, | 24 | 28, | 7 | 8 | 9 | 18, | 25 | 31, |
| V2 | | | | X1 = F | | X1 = F | | | | X1 = S, | | X1 = S, |
| | | | | | | | | | | X2 = N, | | X2 = N, |
| | | | | | | | | | | X3 = F | | X3 = F |
| huC1C1- | 1 | 2 | 3 | 14, | 24 | 27, | 7 | 8 | 9 | 19, | 25 | 32, |
| V3 | | | | X1 = C | | X1 = C | | | | X1 = A, | | X1 = A, |
| | | | | | | | | | | X2 = N, | | X2 = N, |
| | | | | | | | | | | X3 = F | | X3 = F |
| huC1C1- | 1 | 2 | 3 | 14, | 24 | 27, | 7 | 8 | 9 | 20, | 25 | 33, |
| V4 | | | | X1 = C | | X1 = C | | | | X1 = S, | | X1 = S, |
| | | | | | | | | | | X2 = Y, | | X2 = Y, |
| | | | | | | | | | | X3 = F | | X3 = F |
| huC1C1- | 1 | 2 | 3 | 14, | 24 | 27, | 7 | 8 | 9 | 21, | 25 | 34, |
| V5 | | | | X1 = C | | X1 = C | | | | X1 = S, | | X1 = S, |
| | | | | | | | | | | X2 = N, | | X2 = N, |
| | | | | | | | | | | X3 = Y | | X3 = Y |
| huC1C1- | 1 | 2 | 3 | 14, | 24 | 27, | 7 | 8 | 9 | 22, | 25 | 35, |
| V6 | | | | X1 = C | | X1 = C | | | | X1 = A, | | X1 = A, |
| | | | | | | | | | | X2 = Y, | | X2 = Y, |
| | | | | | | | | | | X3 = Y | | X3 = Y |
| huC1C1- | 1 | 2 | 3 | 15, | 24 | 28, | 7 | 8 | 9 | 22, | 25 | 35, |
| V7 | | | | X1 = F | | X1 = F | | | | X1 = A, | | X1 = A, |
| | | | | | | | | | | X2 = Y, | | X2 = Y, |
| | | | | | | | | | | X3 = Y | | X3 = Y |
| Mouse | 4 | 5 | 6 | 16 | 24 | 29 | 10 | 11 | 12 | 23, | 25 | 36, |
| 1B2 | | | | | | | | | | X1 = M | | X1 = M |
| | | | | | | | | | | X2 = N, | | X2 = N, |
| | | | | | | | | | | X3 = A | | X3 = A |

TABLE 2
Amino acid sequences and ID numbers of antibody CDRs defined by difffent
numbering systems/methods

| Anti- | Numbering | | | | | | |
|---|---|---|---|---|---|---|---|
| body | system/ | | | | | | |
| ID | method | VH-CDR1 | VH-CDR2 | VH-CDR3 | VL-CDR1 | VL-CDR2 | VL-CDR3 |
| C1C1 | Kabat | NYGMS | SISSGGDDTYY | AGGKAMDY | RASQSIRDNLH | YASQSIS | QQFNSWPLT |
| | | (SEQ ID | PDNVKG | (SEQ ID | (SEQ ID | (SEQ ID | (SEQ ID |
| | | NO: 1) | (SEQ ID | NO: 3) | NO: 7) | NO: 8) | NO: 9) |
| | | | NO: 2) | | | | |
| | Chothia | GFTFSNY | SSGGDD | AGGKAMDY | RASQSIRDNLH | YASQSIS | QQFNSWPLT |
| | | (SEQ ID | (SEQ ID | (SEQ ID | (SEQ ID | (SEQ ID | (SEQ ID |
| | | NO: 37) | NO: 38) | NO: 3) | NO: 7) | NO: 8) | NO: 9) |
| | IMGT | GFTFSNYG | ISSGGDDT | TRAGGKAMDY | QSIRDN | YAS | QQFNSWPLT |
| | | (SEQ ID | (SEQ ID | (SEQ ID | (SEQ ID | (SEQ ID | (SEQ ID |
| | | NO: 39) | NO: 40) | NO: 41) | NO:57) | NO: 58) | NO: 9) |
| | AbM | GFTFSNYGMS | SISSGGDDTY | AGGKAMDY | RASQSIRDNLH | YASQSIS | QQFNSWPLT |
| | | (SEQ ID | (SEQ ID | (SEQ ID | (SEQ ID | (SEQ ID | (SEQ ID |
| | | NO: 42) | NO: 43) | NO: 3) | NO: 7) | NO: 8) | NO: 9) |
| | Contact | SNYGMS | WVASISSGGDTY | TRAGGKAMD | RDNLHWY | LLINYASQSI | QQFNSWPL |
| | | (SEQ ID | (SEQ ID | (SEQ ID | (SEQ ID | (SEQ ID | (SEQ ID |
| | | NO: 44) | NO: 45) | NO: 46) | NO: 59) | NO: 60) | NO: 61) |
| 1B2 | Kabat | DYVMH | YINPYNDGTKY | GFLRESWFGY | RSSQNIVHSN | KVSNRFS | FQGSHVPPT |
| | | (SEQ ID | NEKFKG | (SEQ ID | GNTYLD | (SEQ ID | (SEQ ID |
| | | NO: 4) | (SEQ ID | NO: 6) | (SEQ ID | NO: 11) | NO: 12) |
| | | | NO: 5) | | NO: 10) | | |
| | Chothia | GYTFTDY | NPYNDG | GFLRESWFGY | RSSQNIVHSN | KVSNRFS | FQGSHVPPT |
| | | (SEQ ID | (SEQ ID | (SEQ ID | GNTYLD | (SEQ ID | (SEQ ID |
| | | NO: 47) | NO: 48) | NO: 6) | (SEQ ID | NO: 11) | NO: 12) |
| | | | | | NO: 10) | | |
| | IMGT | GYTFTDYV | INPYNDGT | ARGFLR.ESW | QNIVHSNGNTY | KVS | FQGSHVPPT |
| | | (SEQ ID | (SEQ ID | FGY | (SEQ ID | (SEQ ID | (SEQ ID |
| | | NO: 49) | NO: 50) | (SEQ ID | NO: 62) | NO: 63) | NO: 12) |
| | | | | NO: 51) | | | |
| | AbM | GYTFTDYVMH | YINPYNDGTK | GFLRESWFGY | RSSQNIVHSN | KVSNRFS | FQGSHVPPT |
| | | (SEQ ID | (SEQ ID | (SEQ ID | GNTYLD | (SEQ ID | (SEQ ID |
| | | NO: 52) | NO: 53) | NO: 6) | (SEQ ID | NO: 11) | NO: 12) |
| | | | | | NO: 10) | | |
| | Contact | TDYVMH | CIGYINPYNDG | ARGFLRESWFG | VHSNGNTYL | LLIYKVSN | FQGSHVPP |
| | | (SEQ ID | TK | (SEQ ID | DWY | RF | (SEQ ID |
| | | NO: 54) | (SEQ ID | NO: 56) | (SEQ ID | (SEQ ID | NO: 66) |
| | | | NO: 55) | | NO: 64) | NO: 65) | |

The V_{H} and V_{L} sequences (or CDR sequences) of other anti-CD40 antibodies which bind to human CD40 can be “mixed and matched” with the V_{H} and V_{L} sequences (or CDR sequences) of the anti-CD40 antibody of the present invention. Preferably, when V_{H} and V_{L} chains (or the CDRs within such chains) are mixed and matched, a V_{H} sequence from a particular V_{H}/V_{L} pairing is replaced with a structurally similar V_{H} sequence. Likewise, preferably a V_{L} sequence from a particular V_{H}/V_{L} pairing is replaced with a structurally similar V_{L} sequence.

Accordingly, in one embodiment, an antibody of the invention, or an antigen binding portion thereof, comprises:

(a) a heavy chain variable region comprising an amino acid sequence listed above in Table 1; and
(b) a light chain variable region comprising an amino acid sequence listed above in Table 1, or the V_{L} of another anti-CD40 antibody, wherein the antibody specifically binds human CD40.

In another embodiment, an antibody of the invention, or an antigen binding portion thereof, comprises:

(a) the CDR1, CDR2, and CDR3 regions of the heavy chain variable region listed above in Table 1 or Table 2; and
(b) the CDR1, CDR2, and CDR3 regions of the light chain variable region listed above in Table 1 or Table 2, or the CDRs of another anti-CD40 antibody, wherein the antibody specifically binds human CD40.

In yet another embodiment, the antibody, or the antigen binding portion thereof, includes the heavy chain variable CDR2 region of the anti-CD40 antibody combined with CDRs of other antibodies which bind human CD40, e.g., CDR1 and/or CDR3 from the heavy chain variable region, and/or CDR1, CDR2, and/or CDR3 from the light chain variable region of a different anti-CD40 antibody.

In addition, it is well known in the art that the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, e.g., Klimka et al., British J. of Cancer 83(2):252-260 (2000); Beiboer et al., J. Mol. Biol. 296:833-849 (2000); Rader et al., Proc. Natl. Acad. Sci. U.S.A. 95:8910-8915 (1998); Barbas et al., J. Am. Chem. Soc. 116:2161-2162 (1994); Barbas et al., Proc. Natl. Acad. Sci. U.S.A. 92:2529-2533 (1995); Ditzel et al., J. Immunol. 157:739-749 (1996); Berezov et al., BIAjournal 8: Scientific Review 8 (2001); Igarashi et al., J. Biochem (Tokyo) 117:452-7 (1995); Bourgeois et al., J. Virol 72:807-10 (1998); Levi et al., Proc. Natl. Acad. Sci. U.S.A. 90:4374-8 (1993); Polymenis and Stoller, J. Immunol. 152:5218-5329 (1994) and Xu and Davis, Immunity 13:37-45 (2000). See also, U.S. Pat. Nos. 6,951,646; 6,914,128; 6,090,382; 6,818,216; 6,156,313; 6,827,925; 5,833,943; 5,762,905 and 5,760,185. Each of these references is hereby incorporated by reference in its entirety.

Accordingly, in another embodiment, antibodies of the invention comprise the CDR2 of the heavy chain variable region of the anti-CD40 antibody and at least the CDR3 of the heavy and/or light chain variable region of the anti-CD40 antibody, or the CDR3 of the heavy and/or light chain variable region of another anti-CD40 antibody, wherein the antibody is capable of specifically binding to human CD40. These antibodies preferably (a) compete for binding with CD40; (b) retain the functional characteristics; and/or (c) have a similar binding affinity as the anti-CD40 antibody of the present invention. In yet another embodiment, the antibodies further may comprise the CDR2 of the light chain variable region of the anti-CD40 antibody, or the CDR2 of the light chain variable region of another anti-CD40 antibody, wherein the antibody is capable of specifically binding to human CD40. In another embodiment, the antibodies of the invention may include the CDR1 of the heavy and/or light chain variable region of the anti-CD40 antibody, or the CDR1 of the heavy and/or light chain variable region of another anti-CD40 antibody, wherein the antibody is capable of specifically binding to human CD40.

Conservative Modifications

In another embodiment, an antibody of the invention comprises a heavy and/or light chain variable region sequences of CDR1, CDR2 and CDR3 sequences which differ from those of the anti-CD40 antibodies of the present invention by one or more conservative modifications. It is understood in the art that certain conservative sequence modification can be made which do not remove antigen binding. See, e.g., Brummell et al., (1993) Biochem 32:1180-8; de Wildt et al., (1997) Prot. Eng. 10:835-41; Komissarov et al., (1997) J. Biol. Chem. 272:26864-26870; Hall et al., (1992) J. Immunol. 149:1605-12; Kelley and O'Connell (1993) Biochem. 32:6862-35; Adib-Conquy et al., (1998) Int. Immunol. 10:341-6 and Beers et al., (2000) Clin. Can. Res. 6:2835-43.

Accordingly, in one embodiment, the antibody comprises a heavy chain variable region comprising CDR1, CDR2, and CDR3 sequences and/or a light chain variable region comprising CDR1, CDR2, and CDR3 sequences, wherein:

(a) the heavy chain variable region CDR1 sequence comprises a sequence listed in Table 1 or Table 2 above, and/or conservative modifications thereof; and/or
(b) the heavy chain variable region CDR2 sequence comprises a sequence listed in Table 1 or Table 2 above, and/or conservative modifications thereof; and/or
(c) the heavy chain variable region CDR3 sequence comprises a sequence listed in Table 1 or Table 2 above, and conservative modifications thereof; and/or
(d) the light chain variable region CDR1, and/or CDR2, and/or CDR3 sequences comprise the sequence(s) listed in Table 1 or Table 2 above; and/or conservative modifications thereof; and
(e) the antibody specifically binds human CD40.

The antibody of the present invention possesses one or more of the following functional properties described above, such as high affinity binding to human CD40, and blocking activity on the binding of CD40 to CD40L, as well as the ability to induce ADCC or CDC against CD40-expressing cells.

In various embodiments, the antibody can be, for example, a mouse, human, humanized or chimeric antibody.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the functions set forth above) using the functional assays described herein.

Engineered and Modified Antibodies

Antibodies of the invention can be prepared using an antibody having one or more of the V_{H}/V_{L} sequences of the anti-CD40 antibody of the present invention as starting material to engineer a modified antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

In certain embodiments, CDR grafting can be used to engineer variable regions of antibodies. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., (1998) Nature 332:323-327; Jones et al., (1986) Nature 321:522-525; Queen et al., (1989) Proc. Natl. Acad. See also U.S.A. 86:10029-10033; U.S. Pat. Nos. 5,225,539; 5,530,101; 5,585,089; 5,693,762 and 6,180,370).

Accordingly, another embodiment of the invention pertains to an isolated monoclonal antibody, or antigen binding portion thereof, comprising a heavy chain variable region comprising CDR1, CDR2, and CDR3 sequences comprising the sequences of the present invention, as described above, and/or a light chain variable region comprising CDR1, CDR2, and CDR3 sequences comprising the sequences of the present invention, as described above. While these antibodies contain the V_{H} and V_{L} CDR sequences of the monoclonal antibody of the present invention, they can contain different framework sequences.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat et al., (1991), cited supra; Tomlinson et al., (1992) J. Mol. Biol. 227:776-798; and Cox et al., (1994) Eur. J. Immunol. 24:827-836; the contents of each of which are expressly incorporated herein by reference. As another example, the germline DNA sequences for human heavy and light chain variable region genes can be found in the Genbank database. For example, the following heavy chain germline sequences found in the HCo7 HuMAb mouse are available in the accompanying Genbank Accession Nos.: 1-69 (NG—0010109, NT—024637 & BC070333), 3-33 (NG—0010109 & NT—024637) and 3-7 (NG—0010109 & NT—024637). As another example, the following heavy chain germline sequences found in the HCo12 HuMAb mouse are available in the accompanying Genbank Accession Nos.: 1-69 (NG—0010109, NT—024637 & BC070333), 5-51 (NG—0010109 & NT—024637), 4-34 (NG—0010109 & NT—024637), 3-30.3 (CAJ556644) & 3-23 (AJ406678).

Antibody protein sequences are compared against a compiled protein sequence database using one of the sequence similarity searching methods called the Gapped BLAST (Altschul et al., (1997), supra), which is well known to those skilled in the art.

Preferred framework sequences for use in the antibodies of the invention are those that are structurally similar to the framework sequences used by antibodies of the invention. The V_{H} CDR1, CDR2, and CDR3 sequences can be grafted onto framework regions that have the identical sequence as that found in the germline immunoglobulin gene from which the framework sequence derives, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

Another type of variable region modification is to mutate amino acid residues within the V_{H} and/or V_{L} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as known in the art. Preferably conservative modifications (as known in the art) are introduced. The mutations can be amino acid substitutions, additions or deletions, but are preferably substitutions. Moreover, typically no more than one, two, three, four or five residues within a CDR region are altered.

Accordingly, in another embodiment, the invention provides isolated anti-CD40 monoclonal antibodies, or antigen binding portions thereof, comprising a heavy chain variable region comprising: (a) a V_{H} CDR1 region comprising the sequence of the present invention, or an amino acid sequence having one, two, three, four or five amino acid substitutions, deletions or additions; (b) a V_{H} CDR2 region comprising the sequence of the present invention, or an amino acid sequence having one, two, three, four or five amino acid substitutions, deletions or additions; (c) a V_{H} CDR3 region comprising the sequence of the present invention, or an amino acid sequence having one, two, three, four or five amino acid substitutions, deletions or additions; (d) a V_{L} CDR1 region comprising the sequence of the present invention, or an amino acid sequence having one, two, three, four or five amino acid substitutions, deletions or additions; (e) a V_{L} CDR2 region comprising the sequence of the present invention, or an amino acid sequence having one, two, three, four or five amino acid substitutions, deletions or additions; and (f) a V_{L} CDR3 region comprising the sequence of the present invention, or an amino acid sequence having one, two, three, four or five amino acid substitutions, deletions or additions.

Engineered antibodies of the invention include those in which modifications have been made to framework residues within V_{H} and/or V_{L}, e.g. to improve the properties of the antibody. Typically, such framework modifications are made to decrease the immunogenicity of the antibody. For example, one approach is to “backmutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043.

In addition, or as an alternative to modifications made within the framework or CDR regions, antibodies of the invention can be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention can be chemically modified (e.g., one or more chemical moieties can be attached to the antibody) or be modified to alter its glycosylation, again to alter one or more functional properties of the antibody.

In one embodiment, the hinge region of C_{H1} is modified in such that the number of cysteine residues in the hinge region is altered, e.g., increased or decreased. This approach is described further in U.S. Pat. No. 5,677,425. The number of cysteine residues in the hinge region of C_{H1} is altered to, for example, facilitate assembly of the light and heavy chains or to change the stability of the antibody.

In another embodiment, the Fc hinge region of an antibody is mutated to decrease the biological half-life of the antibody. More specifically, one or more amino acid mutations are introduced into the C_{H2}-C_{H3} domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In still another embodiment, the glycosylation of an antibody is modified. For example, an aglycosylated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in U.S. Pat. Nos. 5,714,350 and 6,350,861.

Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (α(1,6)-fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see U.S. Patent Publication No. 20040110704 and Yamane-Ohnuki et al., (2004) Biotechnol Bioeng 87:614-22). As another example, EP 1,176,195 describes a cell line with a functionally disrupted FUT8 gene, which encodes a fucosyl transferase, such that antibodies expressed in such a cell line exhibit hypofucosylation by reducing or eliminating the α-1,6 bond-related enzyme. EP 1,176,195 also describes cell lines which have a low enzyme activity for adding fucose to the N-acetylglucose amine that binds to the Fc region of the antibody or does not have the enzyme activity, for example the rat myeloma cell line YB2/0 (ATCC CRL 1662). PCT Publication WO 03/035835 describes a variant CHO cell line, Lec13 cells, with reduced ability to attach fucose to Asn(297)-linked carbohydrates, also resulting in hypofucosylation of antibodies expressed in that host cell (see also Shields et al., (2002) J. Biol. Chem. 277:26733-26740). Antibodies with a modified glycosylation profile can also be produced in chicken eggs, as described in PCT Publication WO 06/089231. Alternatively, antibodies with a modified glycosylation profile can be produced in plant cells, such as Lemna. Methods for production of antibodies in a plant system are disclosed in the U.S. patent application corresponding to Alston & Bird LLP attorney docket No. 040989/314911, filed on Aug. 11, 2006. PCT Publication WO 99/54342 describes cell lines engineered to express glycoprotein-modifying glycosyl transferases (e.g., β(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al., (1999) Nat. Biotech. 17:176-180). Alternatively, the fucose residues of the antibody can be cleaved off using a fucosidase enzyme; e.g., the fucosidase α-L-fucosidase removes fucosyl residues from antibodies (Tarentino et al., (1975) Biochem. 14:5516-23).

Another modification of the antibodies herein that is contemplated by this disclosure is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See, e.g., EP 0 154 316 and EP 0 401 384.

Antibody's Physical Properties

Antibodies of the invention can be characterized by their various physical properties, to detect and/or differentiate different classes thereof.

For example, antibodies can contain one or more glycosylation sites in either the light or heavy chain variable region. Such glycosylation sites may result in increased immunogenicity of the antibody or an alteration of the pK of the antibody due to altered antigen binding (Marshall et al (1972) Annu Rev Biochem 41:673-702; Gala and Morrison (2004) J Immunol 172:5489-94; Wallick et al (1988) J Exp Med 168:1099-109; Spiro (2002) Glycobiology 12:43R-56R; Parekh et al (1985) Nature 316:452-7; Mimura et al., (2000) Mol Immunol 37:697-706). Glycosylation has been known to occur at motifs containing an N-X-S/T sequence. In some instances, it is preferred to have an anti-CD40 antibody that does not contain variable region glycosylation. This can be achieved either by selecting antibodies that do not contain the glycosylation motif in the variable region or by mutating residues within the glycosylation region.

In a preferred embodiment, the antibodies do not contain asparagine isomerism sites. The deamidation of asparagine may occur on N-G or D-G sequences and result in the creation of an isoaspartic acid residue that introduces a link into the polypeptide chain and decreases its stability (also known as isoaspartic acid effect).

Each antibody will have a unique isoelectric point (pI), which generally falls in the pH range between 6 and 9.5. The pI for an IgG1 antibody typically falls within the pH range of 7-9.5 and the pI for an IgG4 antibody typically falls within the pH range of 6-8. There is speculation that antibodies with a pI outside the normal range may have some unfolding and instability under in vivo conditions. Thus, it is preferred to have an anti-CD40 antibody that contains a pI value that falls in the normal range. This can be achieved either by selecting antibodies with a pI in the normal range or by mutating charged surface residues.

Nucleic Acid Molecules Encoding Antibodies of the Invention

In another aspect, the invention provides nucleic acid molecules that encode heavy and/or light chain variable regions, or CDRs, of the antibodies of the invention. The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques. A nucleic acid of the invention can be, e.g., DNA or RNA and may or may not contain intronic sequences. In a preferred embodiment, the nucleic acid is a cDNA molecule.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), a nucleic acid encoding such antibodies can be recovered from the gene library.

Preferred nucleic acids molecules of the invention include those encoding the V_{H} and V_{L} sequences of the CD40 monoclonal antibody or the CDRs. Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (C_{H1}, C_{H2} and C_{H3}). The sequences of human heavy chain constant region genes are known in the art and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain C_{H1} constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art and DNA fragments encompassing these regions can be obtained by standard PCR amplification. In preferred embodiments, the light chain constant region can be a kappa or lambda constant region.

To create a scFv gene, the V_{H}- and V_{L}-encoding DNA fragments are operatively linked to another fragment encoding a flexible linker, e.g., encoding the amino acid sequence (Gly4-Ser)3, such that the V_{H} and V_{L} sequences can be expressed as a contiguous single-chain protein, with the V_{L} and V_{H} regions joined by the flexible linker (see e.g., Bird et al., (1988) Science 242:423-426; Huston et al., (1988) Proc. Natl. Acad. Sci. USA 85:5879-5883; McCafferty et al., (1990) Nature 348:552-554).

Production of Monoclonal Antibodies of the Invention

Monoclonal antibodies (mAbs) of the present invention can be produced using the well-known somatic cell hybridization (hybridoma) technique of Kohler and Milstein (1975) Nature 256: 495. Other embodiments for producing monoclonal antibodies include viral or oncogenic transformation of B lymphocytes and phage display techniques. Chimeric or humanized antibodies are also well known in the art. See e.g., U.S. Pat. Nos. 4,816,567; 5,225,539; 5,530,101; 5,585,089; 5,693,762 and 6,180,370, the contents of which are specifically incorporated herein by reference in their entirety.

Generation of Transfectomas Producing Monoclonal Antibodies of the Invention

Antibodies of the invention also can be produced in a host cell transfectoma using, for example, a combination of recombinant DNA techniques and gene transfection methods as is well known in the art (e.g., Morrison, S. (1985) Science 229:1202). In one embodiment, DNA encoding partial or full-length light and heavy chains obtained by standard molecular biology techniques is inserted into one or more expression vectors such that the genes are operatively linked to transcriptional and translational regulatory sequences. In this context, the term “operatively linked” is intended to mean that an antibody gene is ligated into a vector such that transcriptional and translational control sequences within the vector serve their intended function of regulating the transcription and translation of the antibody gene.

The term “regulatory sequence” is intended to include promoters, enhancers and other expression control elements (e.g., polyadenylation signals) that control the transcription or translation of the antibody genes. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyoma. Alternatively, nonviral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

The antibody light chain gene and the antibody heavy chain gene can be inserted into the same or separate expression vectors. In preferred embodiments, the variable regions are used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes and regulatory sequences, the recombinant expression vectors of the invention can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody.

Preferred mammalian host cells for expressing the recombinant antibodies of the invention include Chinese Hamster Ovary (CHO cells) (including dhfr− CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Immunoconjugates

Antibodies of the invention can be conjugated to a therapeutic agent to form an immunoconjugate such as an antibody-drug conjugate (ADC). Suitable therapeutic agents include cytotoxins, alkylating agents, DNA minor groove binders, DNA intercalators, DNA crosslinkers, histone deacetylase inhibitors, nuclear export inhibitors, proteasome inhibitors, topoisomerase I or II inhibitors, heat shock protein inhibitors, tyrosine kinase inhibitors, antibiotics, and anti-mitotic agents. In the ADC, the antibody and therapeutic agent preferably are conjugated via a linker cleavable such as a peptidyl, disulfide, or hydrazone linker. The ADCs can be prepared as described in U.S. Pat. Nos. 7,087,600; 6,989,452; and 7,129,261; PCT Publications WO 02/096910; WO 07/038,658; WO 07/051,081; WO 07/059,404; WO 08/083,312; and WO 08/103,693; U.S. Patent Publications 20060024317; 20060004081; and 20060247295; the disclosures of which are incorporated herein by reference.

Bispecific Molecules

In another aspect, the present disclosure features bispecific molecules comprising one or more antibodies of the invention linked to at least one other functional molecule, e.g., another peptide or protein (e.g., another antibody or ligand for a receptor) to generate a bispecific molecule that binds to at least two different binding sites or target molecules. Thus, as used herein, “bispecific molecule” includes molecules that have three or more specificities.

In an embodiment, a bispecific molecule has, in addition to an anti-Fc binding specificity and an anti-CD40 binding specificity, a third specificity. The third specificity can be for an anti-enhancement factor (EF), e.g., a molecule that binds to a surface protein involved in cytotoxic activity and thereby increases the immune response against the target cell. For example, the anti-enhancement factor can bind a cytotoxic T-cell (e.g. via CD2, CD3, CD8, CD28, CD4, CD40, or ICAM-1) or other immune cell, resulting in an increased immune response against the target cell.

Bispecific molecules may be in many different formats and sizes. At one end of the size spectrum, a bispecific molecule retains the traditional antibody format, except that, instead of having two binding arms of identical specificity, it has two binding arms each having a different specificity. At the other extreme are bispecific molecules consisting of two single-chain antibody fragments (scFv's) linked by a peptide chain, a so-called Bs(scFv) 2 construct. Intermediate-sized bispecific molecules include two different F(ab) fragments linked by a peptidyl linker. Bispecific molecules of these and other formats can be prepared by genetic engineering, somatic hybridization, or chemical methods. See, e.g., Kufer et al, cited supra; Cao and Suresh, Bioconjugate Chemistry, 9 (6), 635-644 (1998); and van Spriel et al., Immunology Today, 21 (8), 391-397 (2000), and the references cited therein.

Antibody-Encoding or Antibody-Bearing Oncolytic Virus

An oncolytic virus preferentially infects and kills cancer cells. Antibodies of the present invention can be used in conjunction with oncolytic viruses. Alternatively, oncolytic viruses encoding antibodies of the present invention can be introduced into human body.

Pharmaceutical Compositions

In another aspect, the present disclosure provides a pharmaceutical composition comprising one or more antibodies (or antigen-binding portions thereof, or the immunoconjugates, or the bispecifics, or oncolytic viruses,) of the present invention formulated together with a pharmaceutically acceptable carrier. The antibodies (or antigen-binding portions thereof, or the immunoconjugates, or the bispecifics, or oncolytic viruses,) can be dosed separately when the composition contains more than one antibody (or antigen-binding portions thereof, or the immunoconjugates, or the bispecifics, or oncolytic viruses,). The composition may optionally contain one or more additional pharmaceutically active ingredients, such as another antibody or a drug, such as an anti-tumor drug.

The pharmaceutical composition can comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003), the disclosure of which is incorporated herein by reference.

Preferably, the pharmaceutical composition is suitable for intravenous, intramuscular, subcutaneous, parenteral, spinal or epidermal administration (e.g., by injection or infusion). Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the invention can be administered via a non-parenteral route, such as a topical, epidermal or mucosal route of administration, e.g., intranasally, orally, vaginally, rectally, sublingually or topically.

Pharmaceutical compositions can be in the form of sterile aqueous solutions or dispersions. They can also be formulated in a microemulsion, liposome, or other ordered structure suitable to high drug concentration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about ninety-nine percent of active ingredient, preferably from about 0.1% to about 70%, most preferably from about 1% to about 30% of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. Alternatively, antibody can be administered as a sustained release formulation, in which case less frequent administration is required.

For administration of the CD40 antibodies or the pharmaceutical composition comprising the same, the dosage may range from about 0.0001 to 100 mg/kg, and more usually 0.01 to 5 mg/kg, of the host body weight. In some methods, dosage is adjusted to achieve a plasma antibody concentration of about 1-1000 μg/ml and in some methods about 25-300 μg/ml.

A “therapeutically effective dosage” of an anti-CD40 antibody, or the antigen-binding portion thereof, or immunoconjugates, or the bispecifics, or oncolytic viruses, of the invention preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective dosage” preferably inhibits tumor growth by at least about 20%, more preferably by at least about 40%, even more preferably by at least about 60%, and still more preferably by at least about 80% relative to untreated subjects. A therapeutically effective amount of a therapeutic antibody can decrease tumor size, or otherwise ameliorate symptoms in a subject, which is typically a human or can be another mammal.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparatuses (U.S. Pat. Nos. 4,447,233 and 4,447,224); and (5) osmotic devices (U.S. Pat. Nos. 4,439,196 and 4,475,196); the disclosures of which are incorporated herein by reference.

In certain embodiments, the monoclonal antibodies of the invention can be formulated to ensure proper distribution in vivo. For example, to ensure that the therapeutic antibody of the invention cross the blood-brain barrier, they can be formulated in liposomes, which may additionally comprise targeting moieties to enhance selective transport to specific cells or organs. See, e.g. U.S. Pat. Nos. 4,522,811; 5,374,548; 5,416,016; and 5,399,331; V. V. Ranade (1989) J. Clin. Pharmacol. 29:685; Umezawa et al., (1988) Biochem. Biophys. Res. Commun. 153:1038; Bloeman et al., (1995) FEBS Lett. 357:140; M. Owais et al., (1995) Antimicrob. Agents Chemother. 39:180; Briscoe et al., (1995) Am. J. Physiol. 1233:134; Schreier et al., (1994) J. Biol. Chem. 269:9090; Keinanen and Laukkanen (1994) FEBS Lett. 346:123; and Killion and Fidler (1994) Immunomethods 4:273.

Uses and Methods of the Invention

The composition comprising the antibodies or the antigen-binding portion thereof, or immunoconjugates, or the bispecific, or oncolytic viruses, of the present invention have numerous in vitro and in vivo utilities involving, for example, treatment and/or prevention of cancers, inflammatory diseases, or infectious diseases. The antibodies can be administered to human subjects, e.g., in vivo, to inhibit tumor growth.

Given the ability of anti-CD40 antibodies of the invention to inhibit proliferation and survival of cancer cells, the invention provides methods for inhibiting growth of tumor cells in a subject comprising administering to the subject the composition of the invention such that growth of the tumor is inhibited in the subject. Non-limiting examples of tumors that can be treated by antibodies of the invention include, but not limited to, B cell lymphoma, chronic lymphocytic leukemia, multiple myeloma, melanoma, colon adenocarcinoma, pancreas cancer, colon cancer, gastric intestine cancer, prostate cancer, bladder cancer, kidney cancer, ovary cancer, cervix cancer, breast cancer, lung cancer, and nasopharynx cancer, original and/or metastatic cancers, etc. Additionally, refractory or recurrent malignancies whose growth may be inhibited using the antibodies of the invention.

In another aspect, the invention provides a method for treating an inflammatory disease including atherothrombosis and a respiratory disease, as well as an infectious disease in a subject, comprising administering to the subject a therapeutically effective amount of the antibody, or antigen-binding portion thereof, of the invention. Additional anti-inflammatory agents, antimicrobial and/or antiviral agents or other therapeutical agents can be administered with the antibody, or an antigen-binding portion thereof, of the invention.

Generally speaking, the antibodies or the antigen-binding portions thereof of the invention can be used to enhance an immune response in a subject.

Combination Therapy

In another aspect, the invention provides methods of combination therapy in which the anti-CD40 antibodies, or antigen-binding portions thereof, or immunoconjugates, or the bispecifics, or oncolytic viruses, of the present invention are co-administered with one or more additional antibodies that are effective in inhibiting tumor growth in a subject. In one embodiment, the invention provides a method for inhibiting tumor growth in a subject comprising administering to the subject an anti-CD40 antibody (or an antigen-binding portion thereof, or immunoconjugate, or a bispecific molecule, or an oncolytic virus,) and one or more additional antibodies, such as an anti-VISTA antibody, an anti-LAG-3 antibody, an anti-PD-L1 antibody, and anti-PD-1 antibody, and/or an anti-CTLA-4 antibody. In certain embodiments, the subject is human.

The CD40 signaling activation can also be further combined with standard cancer treatments. For example, CD40 signaling activation can be combined with CTLA-4 and/or LAG-3 and/or PD-1 blockade, and/or chemotherapeutic regimes. For example, a chemotherapeutic agent can be administered with the anti-CD40 antibodies, which may be a cytotoxic agent. For example, epirubicin, oxaliplatin, and/or 5-FU are administered to patients receiving anti-CD40 therapy.

Optionally, the combination of anti-CD40 and one or more additional antibodies (e.g., anti-CTLA-4 and/or anti-LAG-3 and/or anti-PD-1 antibodies) can be further combined with an immunogenic agent, such as cancerous cells, purified tumor antigens (including recombinant proteins, peptides, and carbohydrate molecules), and cells transfected with genes encoding immune stimulating cytokines (He et al., (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF.

Other therapies that may be combined with anti-CD40 antibody includes, but not limited to, interleukin-2 (IL-2) administration, radiation, surgery, or hormone deprivation.

The combination of therapeutic agents discussed herein can be administered concurrently as a single composition in a pharmaceutically acceptable carrier, or concurrently as separate compositions with each agent in a pharmaceutically acceptable carrier. In another embodiment, the combination of therapeutic agents can be administered sequentially.

Furthermore, if more than one dose of the combination therapy is administered sequentially, the order of the sequential administration can be reversed or kept in the same order at each time point of administration, sequential administrations can be combined with concurrent administrations, or any combination thereof.

The present disclosure is further illustrated by the following examples, which should not be construed as further limiting. The contents of all figures and all references, Genbank sequences, patents and published patent applications cited throughout this application are expressly incorporated herein by reference.

### EXAMPLES

### Example 1 Generation of Mouse Anti-CD40 Monoclonal Antibodies Using Hybridoma Technology

Immunization

Mice were immunized according to the method as described in E Harlow, D. Lane, Antibody: A Laboratory Manual, Cold Spring Harbor Laboratory Press, Cold Spring Harbor, N.Y., 1998. Recombinant human CD40 protein (AA region 21-193 of Uniprot Number # P25942, amino acid residue 21-193 of SEQ ID NO.: 67) with human IgG1 Fc tag (SEQ ID NO.: 24) at the C-terminus was used as the immunogen. Human CD40-his protein (Acro biosystems, Cat # CD0-H5228) was used for determining anti-sera titer and for screening hybridomas secreting antigen-specific antibodies Immunizing dosages contained 25 μg human CD40-Fc protein/mouse/injection for both primary and boost immunizations. To increase immune response, the complete Freud's adjuvant and incomplete Freud's adjuvant (Sigma, St. Louis, Mo., USA) were used respectively for primary and boost immunizations. Briefly, adjuvant-antigen mixture was prepared by first gently mixing the adjuvant in a vial using a vortex. The desired amount of adjuvant was transferred to an autoclaved 1.5 mL micro-centrifuge tube. The antigen was prepared in PBS or saline with concentration ranging from 0.5-1.0 mg/ml. The calculated amount of antigen was then added to the micro-centrifuge tube with the adjuvant, and the resulting mixture was mixed by gently vortexing for 2 minutes to generate water-in-oil emulsions. The adjuvant-antigen emulsion was then drawn into the proper syringe for animal injection. A total of 25 μg of antigen was injected in a volume of 50-100 μl. Each animal was immunized, and then boosted for 2 to 3 times depending on the anti-sera titer. Animals with good titers were given a final boost by intraperitoneal injection before fusion.

Hybridoma Fusion and Screening

Cells of murine myeloma cell line (SP2/0-Ag14, ATCC # CRL-1581) were cultured to reach the log phase stage right before fusion. Spleen cells from immunized mice were prepared sterilely and fused with myeloma cells according to the method as described in Kohler G, and Milstein C, “Continuous cultures of fused cells secreting antibody of predefined specificity,” Nature, 256: 495-497(1975). Fused “hybrid cells” were subsequently dispensed into 96-well plates in DMEM/20% FCS/HAT media. Surviving hybridoma colonies were observed under the microscope seven to ten days post fusion. After two weeks, the supernatant from each well was subjected to ELISA-based screening using recombinant human CD40-his protein. Briefly, ELISA plates were coated with 60 μl of human CD40-his (Acro biosystems Cat # CD0-H5228, 2.0 μg/ml in PBS) overnight at 4° C. Plates were washed 4 times with PBST and blocked with 200 μl blocking buffer (5% w/v non-fatty milk in PBST). Diluted hybridoma supernatant (60 μl) was added to each well and incubated at 37° C. for 40 minutes. Plates were then washed 4 times, HRP-goat anti-mouse-IgG (Jackson Immuno research, Cat #115-036-071) was added to each well and incubated at 37° C. for 40 minutes. Plates were washed again using wash buffer. TMB was added to each well and incubated at room temperature and away from light for 3-15 min 1M H₂SO₄ solution was added to each well to terminate the reaction Binding ODs were observed at 450 nm. Positive hybridoma secreting antibody that binds to human CD40-his protein were then selected and transferred to 24-well plates. Hybridoma clones producing antibodies that showed high specific binding to in-house prepared 293T-CD40 cells stably expressing human CD40 protein (uniprot # P25942-1, SEQ ID NO.: 67) on the cell membrane, namely Clone C1C1 and 1B2, were subcloned by limiting dilution to ensure the clonality of the cell line, and then monoclonal antibodies were purified. Briefly, Protein A sepharose column (from bestchrom (Shanghai) Biosciences, Cat # AA0273) was washed using PBS buffer in 5 to 10 column volumes. Cell supernatants were passed through the columns, and then the columns were washed using PBS buffer until the absorbance for protein reached the baseline. The columns were eluted with elution buffer (0.1 M Glycine-HCl, pH 2.7), and immediately collected into 1.5 ml tubes with neutralizing buffer (1 M Tris-HCl, pH 9.0). Fractions containing IgG were pooled and dialyzed in PBS overnight at 4° C. Subsequently, the in vitro functional activities of purified monoclonal antibodies were characterized as follows.

### Example 2 Affinity Determination of Mouse Anti-CD40 Monoclonal Antibodies Using BIACORE Surface Plasmon Resonance Technology

The purified anti-CD40 mouse monoclonal antibodies (mAbs) generated in Example 1 were characterized for affinities and binding kinetics by Biacore T200 system (GE healthcare, Pittsburgh, Pa., USA).

Briefly, goat anti-mouse IgG (GE healthcare, Cat # BR100838, Mouse Antibody Capture Kit) was covalently linked to a CM5 or a Protein G chip (carboxy methyl dextran coated chip) via primary amines, using a standard amine coupling kit provided by Biacore (GE healthcare, Pittsburgh, Pa., USA). Un-reacted moieties on the biosensor surface were blocked with ethanolamine. Then, purified anti-CD40 antibodies and two control antibodies, BM1 (Dacetuzumab, Genentech Inc, also referred to as CD40-BM1) and BM2 (Selicrelumab, Abgenix, also referred to as CD40-BM2), at the concentration of 66.7 nM were flowed onto the CM5 chip and the Protein G chip, respectively, at a flow rate of 10 μL/min. Then, human CD40-his (Acro biosystems, Cat # CD0-H5228) or cynomolgus monkey CD40-his protein (Acro biosystems, Cat # CD0-052H6) in HBS EP buffer (provided by Biacore) was flowed onto the chip at a flow rate of 30 μL/min. The antigen-antibody association kinetics was followed for 2 minutes and the dissociation kinetics was followed for 10 minutes. The association and dissociation curves were fit to a 1:1 Langmuir binding model using BIA evaluation software. The K_{D}, Kₐ and K_{d} values were determined and summarized in Table 3.

TABLE 3
Binding affinity of mouse anti-CD40 antibodies

| | Kinetics on Biacore | | | | | |
|---|---|---|---|---|---|---|
| | Human CD40-his | | | Cynomolgus CD40-his | | |
| Mouse | Kₐ | K_{d} | K_{D} | Kₐ | K_{d} | K_{D} |
| mAb | (M⁻¹s⁻¹) | (s⁻¹) | (M) | (M⁻¹s⁻¹) | (s⁻¹) | (M) |
| C1C1 | 2.49E+06 | 5.42E−04 | 2.17E−10 | 2.45E+06 | 1.19E−03 | 4.86E−10 |
| BM1 | 1.50E+05 | 1.66E−02 | 1.11E−08 | / | / | / |
| BM2 | 1.78E+05 | 9.09E−04 | 5.12E−09 | / | / | / |

The antibodies of the present invention specifically bound to human and monkey CD40s, with the antibody C1C1 showing higher binding affinity than BM1 and BM2.

### Example 3 Binding Activity of Mouse Anti-CD40 Monoclonal Antibodies

The binding activities of mouse anti-CD40 antibodies were determined by Capture ELISA and Flow Cytometry (FACS).

For the capture ELISA, 96-well micro plates were coated with 2 μg/ml goat anti-mouse IgG F(ab′)₂ fragment (Jackson Immuno Research, Cat #115-005-072, 100 μl/well) for the binding assay of mouse anti-CD40 antibodies, or goat anti-human IgG F(ab′)₂ fragment (Jackson Immuno Research, Cat #109-005-097, 100 μl/well) for the binding assay of the benchmarks, BM1 and BM2 in PBS and incubated overnight at 4° C. Plates were washed 4 times with wash buffer (PBS+0.05% Tween-20, PBST) and then blocked with 200 μl/well blocking buffer (5% w/v non-fatty milk in PBST) for 2 hours at 37° C. Plates were washed again and incubated with 0.004-66.7 nM (5-fold serial dilution in 2.5% non-fatty milk in PBST) purified mouse anti-CD40 antibodies, BM1, BM2 and negative control (hIgG (human immunoglobulin (pH4), for intravenous injection, Hualan Biological Engineering Inc.), 100 μl/well, for 40 minutes at 37° C., and then washed 4 times again. Plates containing captured anti-CD40 antibodies were incubated with biotin-labeled human CD40-Fc protein (amino acid residue 21-193 of SEQ ID NO: 67 linked to N-terminus of amino acid residue 99-330 of SEQ ID NO.: 19, 1:10000 dilution in 2.5% non-fatty milk in PBST, 100 μl/well) for 40 minutes at 37° C., washed 4 times, and incubated with streptavidin conjugated HRP (1:10000 dilution in PBST, Jackson Immuno Research, Cat #016-030-084, 100 μl/well) for 40 minutes at 37° C. After a final wash, plates were incubated with 100 μl/well ELISA substrate TMB (Innoreagents, Cat # TMB-S-002). The reaction was stopped in 15 minutes at 25° C. with 50 μl/well 1M H₂SO₄, and the absorbance was read at 450 nm and plotted against antibody concentration. Data were analyzed using Graphpad Prism software and EC₅₀ values were reported.

For binding of anti-CD40 antibodies to the surface of 293T-CD40 cells by flow cytometry (FACS), in-house prepared 293T-CD40 cells stably expressing full length human CD40 (uniprot # P25942-1, SEQ ID NO.: 57) on cell membrane were used. The 293T-CD40 cells were prepared by transfecting 293T cells with pCMV-T-P plasmids inserted with CD40 coding sequence between EcoRI and XbalI sites, following the instruction of lipofectamine 300 transfection reagent (Thermo Fisher). In specific, the 293T-CD40 cells were harvested from cell culture flasks, washed two times and resuspended in phosphate buffered saline (PBS) containing 2% v/v Fetal Bovine Serum (FACS buffer). 2×10⁵ cells per well in 96 well-plates were incubated with the mouse anti-CD40 antibodies or the controls of various concentrations in FACS buffer for 40 minutes on ice. Cells were washed twice with FACS buffer, and 100 μL/well R-Phycoerythrin AffiniPure F(ab′)2 Fragment Goat Anti-Mouse IgG (H+L) (1:1000 dilution in FACS buffer, Jackson Immunoresearch, Cat #115-116-146) and 100 μL/well R-Phycoerythrin AffiniPure F(ab′)2 Fragment Goat Anti-Human IgG (1:1000 dilution in FACS buffer, Jackson Immunoresearch, Cat #109-115-098) were added in the wells containing the mouse anti-CD40 antibodies and the benchmarks, respectively. Following an incubation of 40 minutes at 4° C. in dark, cells were washed three times and resuspended in FACS buffer. Fluorescence was measured using a Becton Dickinson FACS Canto II-HTS equipment and plotted against antibody concentration. Data were analyzed using Graphpad Prism software and EC₅₀ values were reported as the antibody concentration to achieve 50% of maximal mouse anti-CD40 antibodies binding to 293T-CD40 cells.

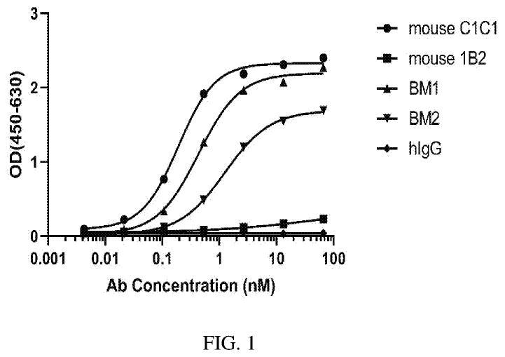

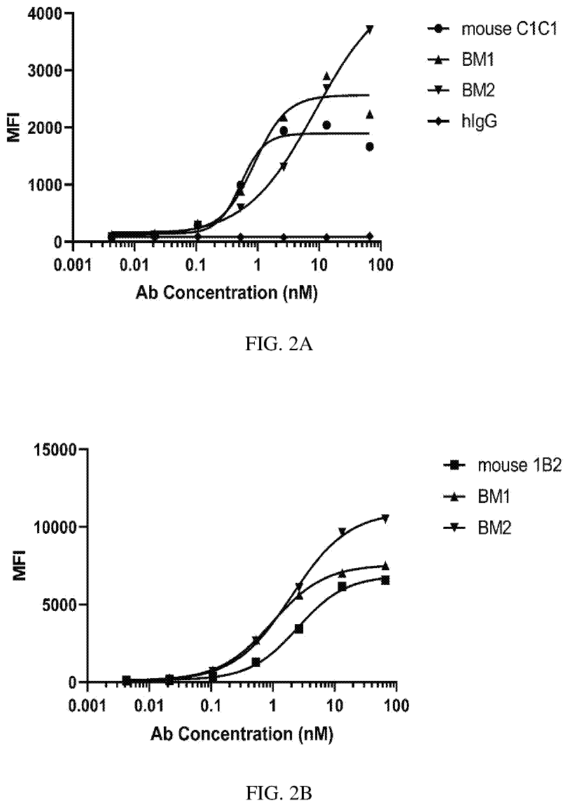

The results were summarized in Table 4 below and shown in FIG. 1 and FIG. 2A-2B.

TABLE 4
Binding Activity of mouse anti-CD40 antibodies

| | FACS cell | Binding capture |
|---|---|---|
| Mouse | binding | ELISA |
| mAb ID | (EC₅₀, nM) | (EC₅₀, nM) |
| 1B2 | 2.538 | 47.11 |
| BM1 | 0.9754 | 0.4337 |
| BM2 | 2.006 | 1.255 |
| C1C1 | 0.5376 | 0.19 |
| BM1 | 0.8860 | 0.4337 |
| BM2 | 8.582 | 1.255 |

The results indicated that the antibodies of the present invention bound to human CD40 specifically, with the antibody C1C1 having lower EC₅₀ values than both of the benchmarks. Further, as can be seen from FIG. 1 and FIG. 2A-2B, the mouse C1C1 antibody showed comparable or better binding capacities than BM1 and BM2, and the mouse 1B2 antibody's binding capacity was comparable to both of the benchmarks.

### Example 4 Functional Assays Using Competitive ELISA and Cell Based Reporter Assay

4.1 Ligand Blocking ELISA

The ability of the mouse anti-CD40 antibodies to block the biding of CD40 to CD40L was measured using a competitive ELISA assay. Briefly, human CD40-Fc proteins (amino acid residue 21-193 of SEQ ID NO: 67 linked to N-terminus of amino acid residue 99-330 of SEQ ID NO.: 24) were coated on 96-well micro plates at 2 μg/mL in coating buffer (PBS) and incubated overnight at 4° C. The next day, plates were washed with wash buffer (PBS+0.05% Tween-20, PBST), and blocked with 5% w/v non-fatty milk in PBST for 2 hours at 37° C. Plates were then washed again using wash buffer.

Serially diluted the mouse anti-CD40 antibodies of the present invention or the controls (starting at 200 nM with a four-fold serial dilution) in 2.5% w/v non-fatty milk in PBST were prepared to the plates and incubated with the human CD40-Fc proteins at room temperature for 40 minutes. Plates were washed 4 times using wash buffer, and then biotin-labeled human CD40L-his protein (1:3500 dilution in 2.5% non-fatty milk in PBST, 100 μl/well, Sino biological Inc., Cat #10239-H08E) was added and incubated for 40 minutes at 37° C. Plates were washed again. 100 μl/well of streptavidin conjugated HRP was added and incubated for 40 minutes at 37° C. Plates were washed again using wash buffer. Finally, TMB was added and the reaction was stopped using 1M H₂SO₄, and the absorbance was read at 450 nm. Data were analyzed using Graphpad Prism software and IC₅₀ values were reported.

4.2 Benchmark Blocking ELISA

The ability of the mouse anti-CD40 antibodies of the present invention to block the binding of the Benchmark to human CD40 was measured using a competitive ELISA assay. Briefly, BM2 was coated on 96-well micro plates at 1 μg/mL in PBS and incubated overnight at 4° C. The next day, plates were washed with wash buffer, and blocked with 5% w/v non-fatty milk in PBST for 2 hours at 37° C. Plates were then washed again using wash buffer. Serially diluted the mouse anti-CD40 antibodies or the controls (starting at 66.7 nM with a 5-fold serial dilution) in biotin labeled human CD40-Fc solution (amino acid residue 21-193 of SEQ ID NO: 67 linked to N-terminus of amino acid residue 99-330 of SEQ ID NO.: 24, 0.23 nM in 2.5% non-fatty milk in PBST) were prepared and incubated at room temperature for 40 minutes, and then mixtures of the antibodies and human CD40-Fc-biotin were added to BM2 coated plates. After incubation at 37° C. for 40 minutes, plates were washed 4 times using wash buffer. Then streptavidin conjugated HRP was added and incubated for 40 minutes at 37° C. to detect biotin-labeled human CD40-Fc bound to plates. Plates were washed again using wash buffer. Finally, TMB was added and the reaction was stopped using 1M H₂SO₄, and the absorbance was read at 450 nm. Data were analyzed using Graphpad Prism software and IC₅₀ values were reported.

4.3 Cell-Based Reporter Assays

The mouse anti-CD40 antibodies were further tested for their agonistic activities using a CD40-expressing reporter cell line 293T-NF-κB-Luc-CD40 which stably express full length human CD40 (uniprot No. P25942-1, SEQ ID NO.: 67). The 293T-NF-κB-Luc-CD40 cells were prepared, following the instruction of lipofectamine 3000 transfection reagent (Thermo Fisher), by transfecting 293T cells with pGL4.32[luc2P NF-κB-RE Hygro] vectors (Promega, GenBank® Accession Number: EU581860) and later pCMV-T-P plasmids inserted with CD40 coding sequence between EcoRI and XbalI sites. When CD40 agonists were added to these cells, CD40 signalings were activated and luciferase expression was upregulated which can be measured by a luminescence assay.

Briefly, 293T-NF-kB-Luc-CD40 cells at the log phase were harvested from cell culture, and resuspended in assay buffer (DMEM medium (Gibco Inc., Cat #10566-016)+10% fetal bovine serum (Gibco Inc., Cat #10099-141)). 5×10³ 293 T-NF-kB-Luc-CD40 cells per well in 384 well-plate were incubated with serially diluted mouse anti-CD40 antibodies or benchmarks (20 μl/well) in assay buffer for 6 hours at 37° C. Then, Luciferase detection Reagent (30 μL/well, Promega, Cat # E6120) was added. Ten minutes later, the plates were subject to analysis by Tecan infinite 200Pro plate-reader. Data of luminescence signal were analyzed using Graphpad prism software and EC₅₀ values were reported.

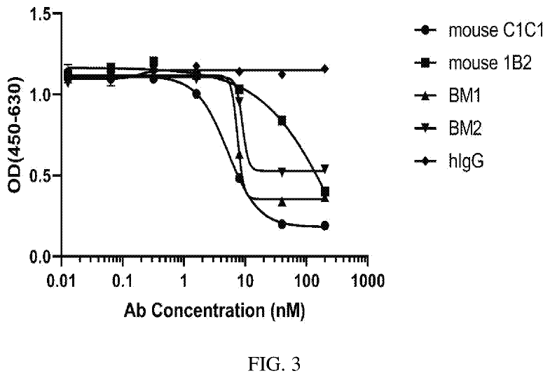

The results of the three assays were summarized in Table 5 below and shown in FIGS. 3, 4A-4B, and 5A-5B.

TABLE 5
Mouse anti-CD40 antibodies' functional assay results

| | Human CD40/human | Benchmark | Cell-based |
|---|---|---|---|
| | CD40L ligand | (BM2)-blocking | Reporter |
| Mouse | blocking ELISA | ELISA | assay |
| mAb ID | (IC₅₀, nM) | (IC₅₀, nM) | (EC₅₀, nM) |
| C1C1 | 5.19 | 0.4607 | 3.653 |
| BM1 | 7.517 | 0.4998 | 4.388 |
| BM2 | 8.998 | 1.860 | 7.013 |
| 1B2 | 315.0 | 4.760 | 6.360 |
| BM1 | 7.517 | Not tested | 4.806 |
| BM2 | 8.998 | 0.8037 | 4.903 |

It can be seen from Table 5 that the anti-CD40 antibody C1C1 was more capable of blocking the binding of human CD40 to human CD40L at lower IC₅₀ value, as compared to the benchmarks. Further, as shown in FIG. 3, mouse C1C1 and 1B2 antibodies had better or comparable blocking activities than the benchmarks, respectively.

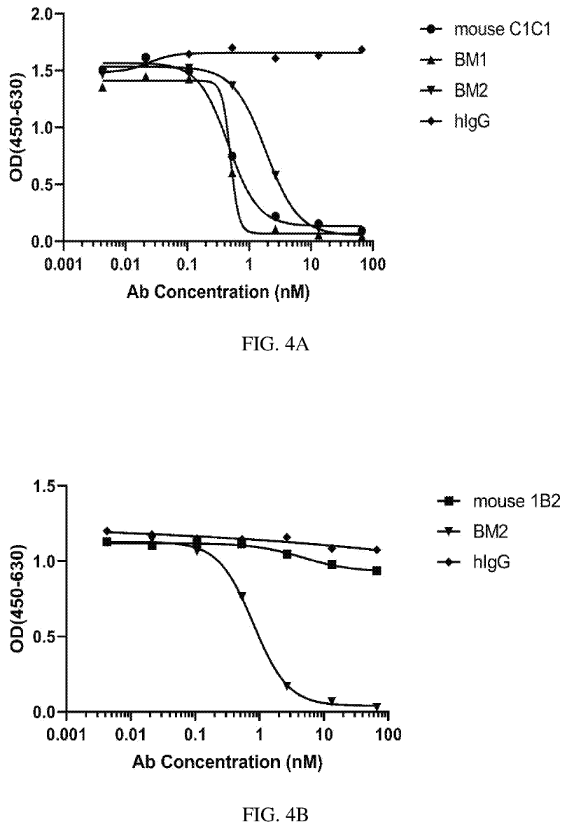

The data also showed that the mouse antibody C1C1 was able to block the binding of human CD40 to BM2 (FIG. 4A), suggesting that there may be some overlaps between the epitopes the mouse antibody C1C1 bound and BM2 did. The mouse antibody 1B2 shows no blocking ability of the binding of human CD40 to BM2 (FIG. 4B), indicated that 1B2 may bind to different epitopes from BM2.

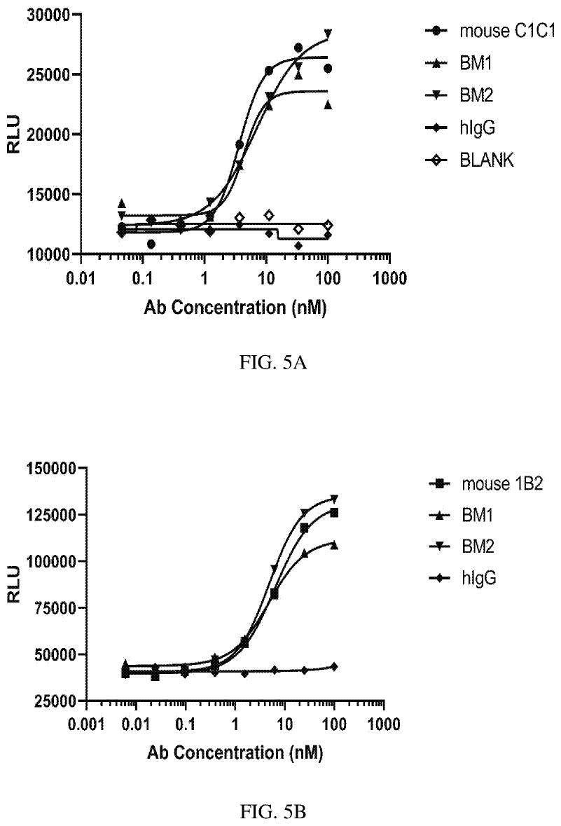

Further, as shown in FIG. 5A-5B, the mouse C1C1 antibody showed higher agonistic activity than the benchmarks, while the mouse 1B2 antibody had comparable agonistic activities compared to BM1 and BM2.

### Example 5 Generation and Characterization of Chimeric Antibodies

The variable domains of the heavy and light chain of the anti-CD40 mouse mAb were sequenced and summarized in Table 1.

The variable domains of the heavy and light chain of the anti-CD40 mouse mAb C1C1 were cloned in frame to the human IgG1 heavy-chain constant region (SEQ ID NO.: 24) and the human kappa light-chain constant region (SEQ ID NO.: 25), respectively. In specific, expression vectors of the chimeric antibody were constructed by inserting the sequence encoding a signal peptide, and the heavy/light chain variable region of the mouse anti-CD40 antibody C1C1 plus human IgG1/kappa constant region into pCEP4, respectively. 293F cells were cultured in serum free medium (Shanghai OPM Biosciences Co. Ltd., OPM-293 CD05) to reach the log phase and transfected with the expression vectors described above using polyethyleneinimine (PEI). Transfected 293F cells were cultured, and cell culture supernatants were harvested and monoclonal antibodies were purified as described in Example 1, the chimeric antibody C1C1 (also called chC1C1) was obtained after purification.

The activities of the resulting chimeric antibody were confirmed in capture ELISA, ligand blocking ELISA, and benchmark blocking ELISA following the protocols in the foregoing Examples. In the capture ELISA test, a mouse control antibody targeting a protein other than CD40 was used.

The data showed that the chimeric and mouse C1C1 antibodies had similar binding and functional activities, as shown in Table 6 below.

TABLE 6
Binding and functional activities of the Chimeric Antibody

| | | Functional activity | |
|---|---|---|---|
| | | hCD40/hCD40L | |
| | Binding | ligand | Benchmark |
| | Capture | blocking | blocking |
| | ELISA | ELISA | ELISA |
| mAb ID# | (EC₅₀, nM) | (IC₅₀, nM) | (IC₅₀, nM) |
| mouse C1C1 | 0.192 | 5.40 | 0.461 |
| chimeric C1C1 | 0.24 | 4.83 | 0.13 |
| BM1 | 0.33 | 6.12 | Not tested |
| BM2 | 0.72 | 10.78 | 0.61 |

### Example 6 Humanization of the Anti-CD40 Mouse Monoclonal Antibody C1C1

The mouse anti-CD40 antibody C1C1 was selected for humanization and further investigations. Humanization of the mouse antibody was conducted using the well-established CDR-grafting method as described in detail below.

To select acceptor frameworks for humanization of the mouse antibody C1C1, the light and heavy chain variable region sequences of mouse C1C1 were blasted against the human immunoglobulin gene database. The human germlines with the highest homology to mouse C1C1 were selected as the acceptor frameworks for humanization, the germlines for the mouse antibody C1C1's VH-CDRs and VL-CDRs being IGHV3-30/JH4d and IGKV3-11/JK1 (NCBI Germline), respectively. The mouse antibody heavy/light chain variable region CDRs were inserted into the selected frameworks, and the residue(s) in the frameworks was/were further mutated to obtain more candidate heavy chain/light chain variable regions. A total of 7 humanized C1C1 antibodies, namely huC1C1-V1 to huC1C1-V7, were obtained whose heavy/light chain variable region sequences were in Table 1.

The vectors containing nucleotide sequences encoding humanized C1C1 heavy chain/light chain variable regions and human IgG1 heavy-chain and human kappa light-chain constant regions (SEQ ID NOs: 72 and 73) were transiently transfected into 50 ml of 293F suspension cell cultures in a ratio of 47.62% to 52.38% light to heavy chain construct, with 1 mg/ml PEI. Cell supernatants were harvested after six days in shaking flasks, spun down to pellet cells, and filtered through 0.22 μm filters for IgG separation. The antibodies were purified by protein A affinity chromatography. Briefly, Protein A sepharose column (from bestchrom (Shanghai) Biosciences, Cat # AA0273) was washed 5 to 10 column volumes using PBS buffer. Cell supernatants were passed through the columns, and then the columns were washed using PBS buffer until the absorbance for protein reached the baseline. The columns were eluted with elution buffer (0.1 M Glycine-HCl, pH 2.7), and immediately collected into 1.5 ml tubes with neutralizing buffer (1 M Tris-HCl, pH 9.0). Fractions containing IgG were pooled and dialyzed in PBS overnight at 4° C.

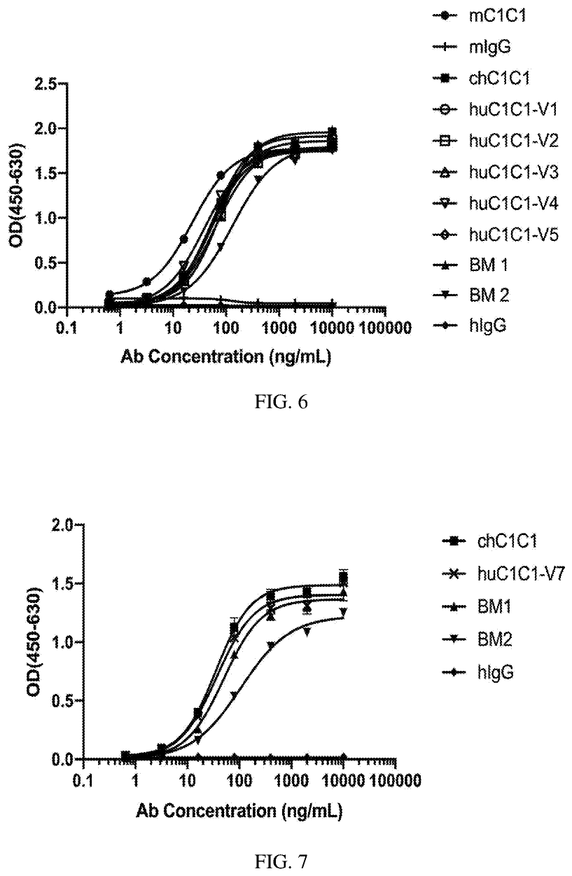

The humanized antibodies huC1C1-V1 to huC1C1-V5 were then tested for the binding ability to human CD40 by binding capture ELISA, and huC1C1-V6 and huC1C1-V7 were tested for the affinity for human CD40 by Biacore, following the protocols in the foregoing Examples. In the capture ELISA, 96-well micro plates were coated with 2 μg/ml goat anti-human IgG F(ab′)₂ fragment (Jackson Immunoresearch, Cat #109-005-097). As shown in Table 7 and FIG. 6, mouse, chimeric and humanized C1C1 antibodies all had higher binding capacities to CD40 than the benchmarks. Further, as shown in Table 8, the humanized antibodies huC1C1-V6 and huC1C1-V7 had higher and comparable affinities to human CD40 as compared to BM1 and BM2, respectively.

TABLE 7
Human CD40 binding capacity of the humanized antibodies huC1C1-V1 to huC1C1-V5

| | | Mouse | | | | | | | | | |
|---|---|---|---|---|---|---|---|---|---|---|---|
| | | IgG-NC | | | | | | | | | |
| Con.of | | (mouse | | | | | | | | | Human |
| mAb | Mouse | control | | huC1C1- | huC1C1- | huC1C1- | huC1C1- | huC1C1- | | | IgG-NC |
| (ng/mL) | C1C1 | Ab) | chC1C1 | V1 | V2 | V3 | V4 | V5 | BM 1 | BM 2 | (hIgG) |
| 10000 | 1.808 | 0.044 | 1.963 | 1.897 | 1.821 | 1.799 | 1.841 | 1.774 | 1.986 | 1.852 | 0.023 |
| 2000 | 1.754 | 0.047 | 1.831 | 1.782 | 1.724 | 1.714 | 1.698 | 1.683 | 1.870 | 1.630 | 0.018 |
| 400 | 1.689 | 0.050 | 1.794 | 1.726 | 1.618 | 1.716 | 1.632 | 1.655 | 1.826 | 1.421 | 0.019 |
| 80 | 1.476 | 0.087 | 1.172 | 1.122 | 1.025 | 1.151 | 1.249 | 1.101 | 1.019 | 0.669 | 0.027 |
| 16 | 0.764 | 0.109 | 0.329 | 0.353 | 0.300 | 0.357 | 0.459 | 0.340 | 0.291 | 0.178 | 0.030 |
| 3.2 | 0.288 | 0.099 | 0.082 | 0.083 | 0.073 | 0.083 | 0.103 | 0.083 | 0.077 | 0.052 | 0.025 |
| 0.64 | 0.142 | 0.096 | 0.035 | 0.035 | 0.029 | 0.032 | 0.039 | 0.033 | 0.036 | 0.028 | 0.026 |
| 0 | 0.124 | 0.125 | 0.028 | 0.028 | 0.027 | 0.024 | 0.025 | 0.027 | 0.028 | 0.029 | 0.026 |
| EC50 | 0.155 | — | 0.384 | 0.382 | 0.428 | 0.336 | 0.267 | 0.355 | 0.501 | 0.886 | — |
| (ng/mL) | | | | | | | | | | | |

TABLE 8
The affinity for human CD40 of the humanized antibodies
huC1C1-V6 and huC1C1-V7 tested by BIAcore

| mAb ID | Ka (1/Ms) | Kd (1/s) | KD (M) |
|---|---|---|---|
| chC1C1 | 8.10E+05 | 0.005745 | 7.09E−09 |
| huC1C1-V6 | 7.63E+05 | 0.008808 | 1.16E−08 |
| huC1C1-V7 | 8.14E+05 | 0.009677 | 1.19E−08 |
| BM1 | 2.79E+05 | 0.007331 | 2.63E−08 |
| BM2 | 1.07E+05 | 0.00165 | 1.54E−08 |

### Example 7. Characterization of Humanized Antibody huC1C1-V7

The humanized anti-CD40 antibody huC1C1-V7 was further tested for their binding and functional activities, following the protocols described in the foregoing Examples. In the FACS test, R-Phycoerythrin AffiniPure Goat Anti-Human IgG Fcγ Fragment Specific (1:1000 dilution in FACS buffer, Jackson Immunoresearch, Cat #109-116-098) was used to test the binding activity of huC1C1-V7. In the cell based reporter assay, anti-CD22 antibody (Epratuzumab, Amgen) and Biotin-human CD40L-his (Cat #10239-H08E, Sino biological Inc.) were used as an isotype control and a positive control, respectively.

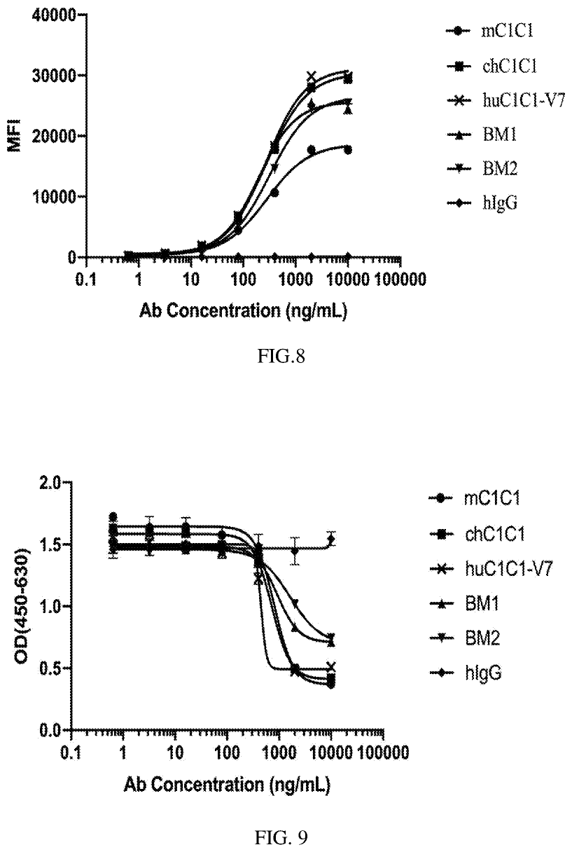

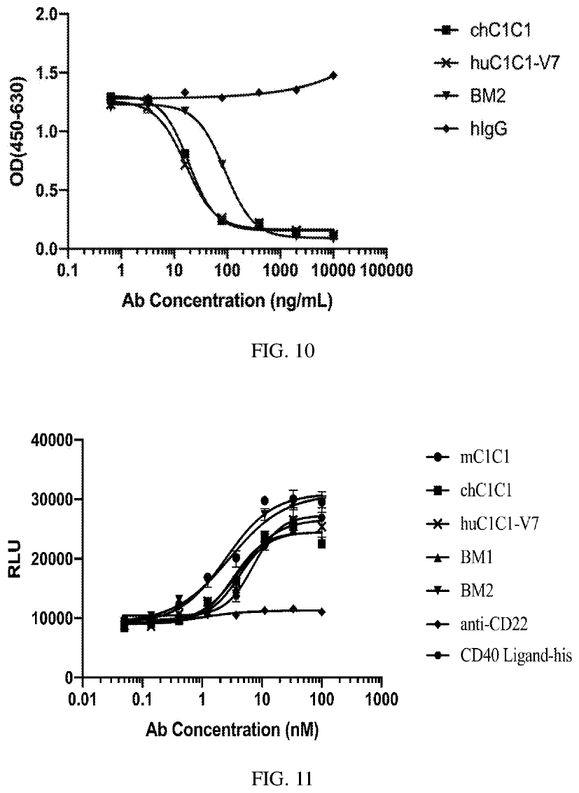

Results were summarized in Table 9 below and shown in FIG. 7-11. The result of the humanized antibody huC1C1-V7 tested by capture ELISA showed good binding activity to human CD40, which was better than the benchmarks (FIG. 7); the antibody huC1C1-V7 can bind to cell surface CD40 efficiently, with a bit higher or comparable binding ability, as compared to BM1 and BM2, respectively (FIG. 8); huC1C1-V7 had also comparable binding affinity to human CD40 and monkey CD40, as compared to the benchmarks; Further, huC1C1-V7 can block the binding of human CD40 to human CD40L more effectively than the benchmarks (FIG. 9), as well as the binding of human CD40 to BM2 (FIG. 10). The humanized antibody huC1C1-V7 showed comparable agonistic activity compared to BM1, although it had a little lower agonistic activity than BM2 (FIG. 11).

TABLE 9
Binding and Functional activities of Humanized mAb huC1C1-V7

| | | | | | Functional assay | | |
|---|---|---|---|---|---|---|---|
| | Binding assay | | | | Competition ELISA (IC₅₀, | | |
| | Human CD40-his | | | | nM) | | |
| | | Cell | | | CD40/CD40L | Benchmark | cell based |
| | Capture | Binding | | Cynomolgus | ligand | blocking | functional |
| | ELISA | FACS | | CD40-his | blocking | ELISA | assay |
| C1C1 | (EC50, | (EC50, | BIAcore | BIAcore | ELISA (IC50, | (IC50, | (EC50, |
| mAbs | nM.) | nM.) | (KD, M) | (KD, M) | nM) | nM) | nM) |
| huC1C1-V7 | 0.248 | 1.846 | 1.13E−08 | 4.04E−09 | 2.999 | 0.111 | 4.248 |
| Chimeric | 0.236 | 1.825 | 5.98E−09 | 2.34E−09 | 4.833 | 0.126 | 3.282 |
| C1C1 | | | | | | | |
| mouse C1C1 | not tested | 1.864 | 3.62E−09 | 2.35E−09 | 5.404 | not tested | 2.506 |
| BM1 | 0.335 | 1.303 | 1.41E−08 | 5.85E−09 | 6.116 | not tested | 4.162 |
| BM2 | 0.716 | 2.11 | 1.22E−08 | 6.03E−09 | 10.78 | 0.608 | 2.893 |

While the invention has been described above in connection with one or more embodiments, it should be understood that the invention is not limited to those embodiments, and the description is intended to cover all alternatives, modifications, and equivalents, as may be included within the spirit and scope of the appended claims. All referenced cited herein are further incorporated by reference in their entirety.

Sequences in the present application are summarized below.

Description/
Sequence/SEQ ID NO.

VH-CDR1 for mouse, chimeric and humanized C1C1 antibodies, defined by kabat
numbering system
NYGMS (SEQ ID NO: 1)
VH-CDR2 for mouse, chimeric and humanized C1C1 antibodies, defined by kabat
numbering system
SISSGGDDTYYPDNVKG (SEQ ID NO: 2)
VH-CDR3 for mouse, chimeric and humanized C1C1 antibodies, defined by kabat, Chothia
or AbM numbering system
AGGKAMDY (SEQ ID NO: 3)
VH-CDR1 for 1B2 antibody, defined by kabat numbering system
DYVMH (SEQ ID NO: 4)
VH-CDR2 for 1B2 antibody, defined by kabat numbering system
YINPYNDGTKYNEKFKG (SEQ ID NO: 5)
VH-CDR3 for 1B2 antibody, defined by kabat, Chothia or AbM numbering system
GFLRESWFGY (SEQ ID NO: 6)
VL-CDR1 for mouse, chimeric and humanized C1C1 antibodies, defined by kabat, Chothia
or AbM numbering system
RASQSIRDNLH (SEQ ID NO: 7)
VL-CDR2 for mouse, chimeric and humanized C1C1 antibodies, defined by kabat, Chothia
or AbM numbering system
YASQSIS (SEQ ID NO: 8)
VL-CDR3 for mouse, chimeric and humanized C1C1 antibodies, defined by kabat, Chothia,
IMGT or AbM numbering system
QQFNSWPLT (SEQ ID NO: 9)
VL-CDR1 for 1B2 antibodies, defined by kabat, Chothia or AbM numbering system
RSSQNIVHSNGNTYLD (SEQ ID NO: 10)
VL-CDR2 for 1B2 antibodies, defined by kabat, Chothia or AbM numbering system
KVSNRFS (SEQ ID NO: 11)
VL-CDR3 for 1B2 antibodies, defined by kabat, Chothia, IMGT or AbM numbering system
FQGSHVPPT (SEQ ID NO: 12)
VH for mouse and chimeric C1C1 antibodies
EVKLVESGGGLVKPGASLKLSCAASGFTFSNYGMSWVRQNSDKRLEWVASISSGG
DDTYYPDNVKGRCTISRENAKNTLYLEMSSLKTEDTALYYCTRAGGKAMDYWGQ
GTSVTVST (SEQ ID NO: 13)
Nucleotides encoding VH for mouse and chimeric C1C1 antibodies
GAAGTGAAGCTGGTGGAGTCTGGGGGAGGCTTAGTGAAGCCTGGAGCGTCTCTG
AAACTCTCCTGTGCAGCCTCTGGATTCACTTTCAGTAACTATGGCATGTCTTGGGT
TCGCCAGAATTCAGACAAGAGGCTGGAGTGGGTCGCATCCATTAGTAGTGGTGG
TGATGACACCTACTATCCAGACAATGTAAAGGGCCGATGCACCATCTCCAGAGA
GAATGCCAAGAACACCCTGTATTTGGAGATGAGTAGTCTGAAGACTGAGGACAC
GGCCTTGTATTACTGTACAAGAGCTGGGGGGAAGGCTATGGACTACTGGGGTCA
AGGAACCTCAGTCACCGTCTCCACA (SEQ ID NO: 68)
VH for huC1C1-V1, huC1C1-V3, huC1C1-V4, huC1C1-V5 and huC1C1-V6
EVQLVESGGGVVQPGRSLRLSCAASGFTFSNYGMSWVRQAPGKGLEWVASISSGG
DDTYYPDNVKGRX1TISRDNSKNTLYLQMNSLRAEDTAVYYCTRAGGKAMDYWG
QGTLVTVSS (SEQ ID NO: 14, X1 = C)
EVQLVESGGGVVQPGRSLRLSCAASGFTFSNYGMSWVRQAPGKGLEWVASISSGG
DDTYYPDNVKGRCTISRDNSKNTLYLQMNSLRAEDTAVYYCTRAGGKAMDYWGQ
GTLVTVSS
VH for huC1C1-V2 and huC1C1-V7
EVQLVESGGGVVQPGRSLRLSCAASGFTFSNYGMSWVRQAPGKGLEWVASISSGG
DDTYYPDNVKGRX1TISRDNSKNTLYLQMNSLRAEDTAVYYCTRAGGKAMDYWG
QGTLVTVSS (SEQ ID NO: 15, X1 = F)
EVQLVESGGGVVQPGRSLRLSCAASGFTFSNYGMSWVRQAPGKGLEWVASISSGG
DDTYYPDNVKGRFTISRDNSKNTLYLQMNSLRAEDTAVYYCTRAGGKAMDYWGQ
GTLVTVSS
VH for 1B2
EVQLQQSGPELVKPGASVKMSCKASGYTFTDYVMHWVKQKPGQGLECIGYINPYN
DGTKYNEKFKGKATLTSDKSSSAAYLELSSLTSEDSAVYYCARGFLRESWFGYWG
QGTLVTVSA (SEQ ID NO: 16)
Nucleotides encoding VH for 1B2
GAGGTCCAGCTGCAGCAGTCTGGACCTGAGCTGGTAAAGCCTGGGGCTTCAGTG
AAGATGTCCTGCAAGGCTTCTGGATACACATTCACTGACTATGTTATGCACTGGG
TGAAGCAGAAGCCTGGGCAGGGCCTTGAGTGTATTGGATATATTAATCCTTACAA
TGATGGTACTAAGTACAATGAGAAGTTCAAAGGCAAGGCCACACTGACTTCAGA
CAAATCCTCCAGCGCAGCCTACCTGGAGCTCAGCAGCCTGACCTCTGAGGACTCT
GCGGTCTATTACTGTGCAAGAGGGTTCCTACGAGAGTCCTGGTTTGGTTACTGGG
GCCAAGGGACTCTGGTCACTGTCTCTGCA (SEQ ID NO: 69)
VL for mouse and chimeric C1C1 antibodies
DIVLTQSPATLSVIPGNSVSLSCRASQSIRDNLHWYQQKSHESPRLLINYASQSISGIP
SRFSGSGSGTDFTLTINSVETEDFGIYFCQQFNSWPLTFGGGTKLELK (SEQ ID NO:
17)
Nucleotides encoding VL for mouse and chimeric C1C1 antibodies
GACATTGTGCTAACTCAGTCTCCAGCCACCCTGTCTGTGATTCCAGGAAATAGCG
TCAGTCTTTCCTGCAGGGCCAGCCAAAGTATTCGCGACAACCTACACTGGTATCA
ACAAAAGTCACATGAGTCTCCAAGGCTTCTCATCAACTATGCTTCCCAGTCCATC
TCTGGGATCCCCTCCCGGTTCAGTGGCAGTGGATCAGGGACAGATTTCACTCTCA
CTATCAACAGTGTGGAGACTGAAGATTTTGGAATATATTTCTGTCAGCAATTTAA
CAGCTGGCCCCTCACGTTCGGTGGTGGGACCAAGCTGGAGCTGAAA (SEQ ID NO:
70)
VL for huC1C1-V1 and huC1C1-V2
EIVLTQSPATLSLSPGERATLSCRASQSIRDNLHWYQQKPGQX1PRLLIX2YASQSISG
IPARFSGSGSGTDFTLTISSLEPEDFAVYX3CQQFNSWPLTFGQGTKVEIK (SEQ ID
NO: 18, X1 = S, X2 = N, X3 = F)
EIVLTQSPATLSLSPGERATLSCRASQSIRDNLHWYQQKPGQSPRLLINYASQSISGIP
ARFSGSGSGTDFTLTISSLEPEDFAVYFCQQFNSWPLTFGQGTKVEIK
VL for huC1C1-V3
EIVLTQSPATLSLSPGERATLSCRASQSIRDNLHWYQQKPGQX1PRLLIX2YASQSISG
IPARFSGSGSGTDFTLTISSLEPEDFAVYX3CQQFNSWPLTFGQGTKVEIK (SEQ ID
NO: 19, X1 = A, X2 = N, X3 = F)
EIVLTQSPATLSLSPGERATLSCRASQSIRDNLHWYQQKPGQAPRLLINYASQSISGIP
ARFSGSGSGTDFTLTISSLEPEDFAVYFCQQFNSWPLTFGQGTKVEIK
VL for huC1C1-V4
EIVLTQSPATLSLSPGERATLSCRASQSIRDNLHWYQQKPGQX1PRLLIX2YASQSISG
IPARFSGSGSGTDFTLTISSLEPEDFAVYX3CQQFNSWPLTFGQGTKVEIK (SEQ ID
NO: 20, X1 = S, X2 = Y, X3 = F)
EIVLTQSPATLSLSPGERATLSCRASQSIRDNLHWYQQKPGQSPRLLIYYASQSISGIP
ARFSGSGSGTDFTLTISSLEPEDFAVYFCQQFNSWPLTFGQGTKVEIK
VL for huC1C1-V5
EIVLTQSPATLSLSPGERATLSCRASQSIRDNLHWYQQKPGQX1PRLLIX2YASQSISG
IPARFSGSGSGTDFTLTISSLEPEDFAVYX3CQQFNSWPLTFGQGTKVEIK (SEQ ID
NO: 21, X1 = S, X2 = N, X3 = Y)
EIVLTQSPATLSLSPGERATLSCRASQSIRDNLHWYQQKPGQSPRLLINYASQSISGIP
ARFSGSGSGTDFTLTISSLEPEDFAVYYCQQFNSWPLTFGQGTKVEIK
VL for huC1C1-V6 and huC1C1-V7
EIVLTQSPATLSLSPGERATLSCRASQSIRDNLHWYQQKPGQX1PRLLIX2YASQSISG
IPARFSGSGSGTDFTLTISSLEPEDFAVYX3CQQFNSWPLTFGQGTKVEIK (SEQ ID
NO: 22, X1 = A, X2 = Y, X3 = Y)
EIVLTQSPATLSLSPGERATLSCRASQSIRDNLHWYQQKPGQAPRLLIYYASQSISGIP
ARFSGSGSGTDFTLTISSLEPEDFAVYYCQQFNSWPLTFGQGTKVEIK
VL for 1B2
DVLX1TQTPLSLPVSLGDQASISCRSSQNIVHSNGNTYLDWYLQKPGQSPKLLIYKV
SNRFSGVPDRFSGSGSGTDFTLKIX2RVEAEDLGVYYCFQGSHVPPTFGX3GTKLEL
K (SEQ ID NO: 23, X1 = M, X2 = N, X3 = A)
DVLMTQTPLSLPVSLGDQASISCRSSQNIVHSNGNTYLDWYLQKPGQSPKLLIYKVS
NRFSGVPDRFSGSGSGTDFTLKINRVEAEDLGVYYCFQGSHVPPTFGAGTKLELK
Nucleotides encoding VL for 1B2
GATGTTTTGATGACCCAAACTCCACTCTCCCTGCCTGTCAGTCTTGGAGATCAAG
CCTCCATCTCTTGCAGATCTAGTCAGAACATTGTACATAGTAATGGAAACACCTA
TTTAGATTGGTACCTGCAGAAACCAGGCCAGTCTCCAAAGCTCCTGATCTACAAA
GTTTCCAACCGATTTTCTGGGGTCCCAGACAGGTTCAGTGGCAGTGGATCAGGGA
CAGATTTCACACTCAAGATCAACAGAGTGGAGGCTGAGGATCTGGGAGTTTATT
ACTGCTTTCAAGGTTCACATGTTCCTCCTACGTTCGGTGCTGGGACCAAGCTGGA
GCTGAAA (SEQ ID NO: 71)
CH, heavy chain constant region
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK (SEQ ID NO.:
24)
Nucleotides encoding CH
GCTTCTACTAAAGGTCCTTCCGTTTTTCCCTTAGCCCCATCATCGAAGAGTACCAG
CGGCGGAACAGCAGCGTTGGGGTGTCTTGTCAAAGATTATTTCCCGGAACCTGTA
ACGGTGTCTTGGAATTCCGGTGCTCTCACTTCAGGCGTTCATACCTTTCCCGCCGT
CCTACAATCGAGTGGACTGTACAGCTTATCTTCCGTAGTGACAGTTCCATCATCG
AGTTTGGGGACGCAGACTTATATTTGCAACGTCAATCACAAGCCGAGCAACACC
AAAGTAGACAAGAAAGTGGAGCCTAAGTCTTGTGATAAAACACATACGTGCCCC
CCATGTCCGGCACCTGAACTTCTCGGTGGCCCCTCCGTTTTCCTATTTCCACCGAA
GCCTAAAGACACTCTGATGATCTCACGTACCCCCGAGGTCACATGCGTAGTGGTT
GATGTCTCGCACGAAGACCCAGAGGTAAAGTTCAATTGGTACGTGGATGGAGTT
GAAGTCCATAACGCGAAAACGAAGCCGCGCGAGGAACAATATAATAGTACTTAC
CGAGTAGTGAGCGTTTTAACCGTCTTGCACCAGGACTGGCTTAACGGGAAAGAG
TATAAGTGTAAAGTATCTAATAAGGCTCTCCCTGCCCCCATAGAAAAAACAATTT
CCAAGGCAAAAGGTCAACCACGGGAGCCGCAGGTGTACACGCTACCTCCCTCAA
GAGAAGAGATGACTAAGAACCAAGTTTCGCTGACCTGCTTAGTCAAAGGCTTTT
ATCCAAGTGATATCGCGGTAGAATGGGAGAGCAATGGACAGCCGGAAAACAATT
ACAAGACAACGCCTCCCGTGTTGGACTCTGATGGGTCCTTCTTTCTTTATTCAAA
ACTCACTGTTGACAAGTCGAGGTGGCAACAGGGTAACGTCTTCAGTTGTAGCGTA
ATGCATGAGGCTCTACACAATCATTACACCCAAAAATCTCTGTCCTTATCACCAG
GCAAG (SEQ ID NO.: 72)
CL, light chain constant region
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC (SEQ ID
NO.: 25)
Nucleotides encoding CL
CGTACGGTGGCGGCGCCATCTGTCTTCATCTTCCCGCCATCTGATGAGCAGTTGA
AATCTGGAACTGCCTCTGTTGTGTGCCTGCTGAATAACTTCTATCCCAGAGAGGC
CAAAGTACAGTGGAAGGTGGATAACGCCCTCCAATCGGGTAACTCCCAGGAGAG
TGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGTTGA
(SEQ ID NO: 73)
Heavy chain for mouse and chimeric C1C1 antibodies
EVKLVESGGGLVKPGASLKLSCAASGFTFSNYGMSWVRQNSDKRLEWVASISSGG
DDTYYPDNVKGRCTISRENAKNTLYLEMSSLKTEDTALYYCTRAGGKAMDYWGQ
GTSVTVSTASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQSKSLSLSPGK
(SEQ ID NO: 26)
Heavy chain for huC1C1-V1, huC1C1-V3, huC1C1-V4, huC1C1-V5 and huC1C1-V6
EVQLVESGGGVVQPGRSLRLSCAASGFTFSNYGMSWVRQAPGKGLEWVASISSGG
DDTYYPDNVKGRX1TISRDNSKNTLYLQMNSLRAEDTAVYYCTRAGGKAMDYWG
QGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDK
THTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK (SEQ
ID NO: 27, X1 = C)
EVQLVESGGGVVQPGRSLRLSCAASGFTFSNYGMSWVRQAPGKGLEWVASISSGG
DDTYYPDNVKGRCTISRDNSKNTLYLQMNSLRAEDTAVYYCTRAGGKAMDYWGQ
GTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
Heavy chain for huC1C1-V2 and huC1C1-V7
EVQLVESGGGVVQPGRSLRLSCAASGFTFSNYGMSWVRQAPGKGLEWVASISSGG
DDTYYPDNVKGRX1TISRDNSKNTLYLQMNSLRAEDTAVYYCTRAGGKAMDYWG
QGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDK
THTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
(SEQ ID NO: 28, X1 = F)
EVQLVESGGGVVQPGRSLRLSCAASGFTFSNYGMSWVRQAPGKGLEWVASISSGG
DDTYYPDNVKGRFTISRDNSKNTLYLQMNSLRAEDTAVYYCTRAGGKAMDYWGQ
GTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
Heavy chain for 1B2
EVQLQQSGPELVKPGASVKMSCKASGYTFTDYVMHWVKQKPGQGLECIGYINPYN
DGTKYNEKFKGKATLTSDKSSSAAYLELSSLTSEDSAVYYCARGFLRESWFGYWG
QGTLVTVSAASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDK
THTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
(SEQ ID NO: 29)
Light chain for mouse and chimeric C1C1 antibodies
DIVLTQSPATLSVIPGNSVSLSCRASQSIRDNLHWYQQKSHESPRLLINYASQSISGIP
SRFSGSGSGTDFTLTINSVETEDFGIYFCQQFNSWPLTFGGGTKLELKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC (SEQ ID NO: 30)
Light chain for huC1C1-V1 and huC1C1-V2
EIVLTQSPATLSLSPGERATLSCRASQSIRDNLHWYQQKPGQX1PRLLIX2YASQSISG
IPARFSGSGSGTDFTLTISSLEPEDFAVYX3CQQFNSWPLTFGQGTKVEIKRTVAAPS
VFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC (SEQ ID NO: 31,
X1 = S, X2 = N, X3 = F)
EIVLTQSPATLSLSPGERATLSCRASQSIRDNLHWYQQKPGQSPRLLINYASQSISGIP
ARFSGSGSGTDFTLTISSLEPEDFAVYFCQQFNSWPLTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Light chain for huC1C1-V3
EIVLTQSPATLSLSPGERATLSCRASQSIRDNLHWYQQKPGQX1PRLLIX2YASQSISG
IPARFSGSGSGTDFTLTISSLEPEDFAVYX3CQQFNSWPLTFGQGTKVEIKRTVAAPS
VFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC (SEQ ID NO: 32,
X1 = A, X2 = N, X3 = F)
EIVLTQSPATLSLSPGERATLSCRASQSIRDNLHWYQQKPGQAPRLLINYASQSISGIP
ARFSGSGSGTDFTLTISSLEPEDFAVYFCQQFNSWPLTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Light chain for huC1C1-V4
EIVLTQSPATLSLSPGERATLSCRASQSIRDNLHWYQQKPGQX1PRLLIX2YASQSISG
IPARFSGSGSGTDFTLTISSLEPEDFAVYX3CQQFNSWPLTFGQGTKVEIKRTVAAPS
VFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC (SEQ ID NO: 33,
VFIFPPSDEQLKSGTASVVCLLNNFYPREAKVQWKVDNALQSGNSQESVTEQDSKDS
X1 = S, X2 = Y, X3 = F)
EIVLTQSPATLSLSPGERATLSCRASQSIRDNLHWYQQKPGQSPRLLIYYASQSISGIP
ARFSGSGSGTDFTLTISSLEPEDFAVYFCQQFNSWPLTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Light chain for huC1C1-V5
EIVLTQSPATLSLSPGERATLSCRASQSIRDNLHWYQQKPGQX1PRLLIX2YASQSISG
IPARFSGSGSGTDFTLTISSLEPEDFAVYX3CQQFNSWPLTFGQGTKVEIKRTVAAPS
VFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC (SEQ ID NO: 34,
X1 = S, X2 = N, X3 = Y)
EIVLTQSPATLSLSPGERATLSCRASQSIRDNLHWYQQKPGQSPRLLINYASQSISGIP
ARFSGSGSGTDFTLTISSLEPEDFAVYYCQQFNSWPLTFGQGTKVEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
Light chain for huC1C1-V6 and huC1C1-V7
EIVLTQSPATLSLSPGERATLSCRASQSIRDNLHWYQQKPGQX1PRLLIX2YASQSISG
IPARFSGSGSGTDFTLTISSLEPEDFAVYX3CQQFNSWPLTFGQGTKVEIKRTVAAPS
VFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC (SEQ ID NO: 35,
X1 = A, X2 = Y, X3 = Y)
EIVLTQSPATLSLSPGERATLSCRASQSIRDNLHWYQQKPGQAPRLLIYYASQSISGIP
ARFSGSGSGTDFTLTISSLEPEDFAVYYCQQFNSWPLTFGQGTKVEIKRTVAAPSVFI
FPPSDEQLKSGTASVVCLLNNFYPREAKVQWKVNDALQSGNSQESVTEQDSKDSTY
SLSSTLTLSKADYEKHKVYACEVTHQGLSSPVTKSFNRGEC
Light chain for 1B2
DVLX1TQTPLSLPVSLGDQASISCRSSQNIVHSNSNTYLDWYLQKPGQSPKLLIYKV
SNRFSGVPDRFSGSGSGTDFTLKIX2RVEAEDLGVYYCFQGSHVPPTFGX3GTKLEL
KRTVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC (SEQ ID
NO: 36, X1 = M, X2 = N, X3 = A)
DVLMTQTPLSLPVSLGDQASISCRSSQNIVHSNGNTYLDWYLQKPGQSPKLLIYKVS
NRFSGVPDRFSGSGSGTDFTLKINRVEAEDLGVYYCFQGSHVPPTFGAGTKLELKRT
VAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
VH-CDR1 for mouse, chimeric and humanized C1C1 antibodies, defined by Chothia
numbering system
GFTFSNY (SEQ ID NO: 37)
VH-CDR2 for mouse, chimeric and humanized C1C1 antibodies, defined by Chothia
numbering system
SSGGDD (SEQ ID NO: 38)
VH-CDR1 for mouse, chimeric and humanized C1C1 antibodies, defined by IMGT
numbering system
GFTFSNYG (SEQ ID NO: 39)
VH-CDR2 for mouse, chimeric and humanized C1C1 antibodies, defined by IMGT
numbering system
ISSGGDDT (SEQ ID NO: 40)
VH-CDR3 for mouse, chimeric and humanized C1C1 antibodies, defined by IMGT
numbering system
TRAGGKAMDY (SEQ ID NO: 41)
VH-CDR1 for mouse, chimeric and humanized C1C1 antibodies, defined by AbM numbering
system
GFTFSNYGMS (SEQ ID NO: 42)
VH-CDR2 for mouse, chimeric and humanized C1C1 antibodies, defined by AbM numbering
system
SISSGGDDTY (SEQ ID NO: 43)
VH-CDR1 for mouse, chimeric and humanized C1C1 antibodies, defined by Contact
numbering system
SNYGMS (SEQ ID NO: 44)
VH-CDR2 for mouse, chimeric and humanized C1C1 antibodies, defined by Contact
numbering system
WVASISSGGDDTY (SEQ ID NO: 45)
VH-CDR3 for mouse, chimeric and humanized C1C1 antibodies, defined by Contact
numbering system
TRAGGKAMD (SEQ ID NO: 46)
VH-CDR1 for 1B2 antibody, defined by Chothia numbering system
GYTFTDY (SEQ ID NO: 47)
VH-CDR2 for 1B2 antibody, defined by Chothia numbering system
NPYNDG (SEQ ID NO: 48)
VH-CDR1 for 1B2 antibody, defined by IMGT numbering system
GYTFTDYV (SEQ ID NO: 49)
VH-CDR2 for 1B2 antibody, defined by IMGT numbering system
INPYNDGT (SEQ ID NO: 50)
VH-CDR3 for 1B2 antibody, defined by IMGT numbering system
ARGFLR.ESWFGY (SEQ ID NO: 51)
VH-CDR1 for 1B2 antibody, defined by AbM numbering system
GYTFTDYVMH (SEQ ID NO: 52)
VH-CDR2 for 1B2 antibody, defined by AbM numbering system
YINPYNDGTK (SEQ ID NO: 53)
VH-CDR1 for 1B2 antibody, defined by Contact numbering system
TDYVMH (SEQ ID NO: 54)
VH-CDR2 for 1B2 antibody, defined by Contact numbering system
CIGYINPYNDGTK (SEQ ID NO: 55)
VH-CDR3 for 1B2 antibody, defined by Contact numbering system
ARGFLRESWFG (SEQ ID NO: 56)
VL-CDR1 for mouse, chimeric and humanized C1C1 antibodies, defined by IMGT
numbering system
QSIRDN (SEQ ID NO: 57)
VL-CDR2 for mouse, chimeric and humanized C1C1 antibodies, defined by IMGT
numbering system
YAS (SEQ ID NO: 58)
VL-CDR1 for mouse, chimeric and humanized C1C1 antibodies, defined by Contact
numbering system
RDNLHWY (SEQ ID NO: 59)
VL-CDR2 for mouse, chimeric and humanized C1C1 antibodies, defined by Contact
numbering system
LLINYASQSI (SEQ ID NO: 60)
VL-CDR3 for mouse, chimeric and humanized C1C1 antibodies, defined by Contact
numbering system
QQFNSWPL (SEQ ID NO: 61)
VL-CDR1 for 1B2 antibody, defined by IMGT numbering system
QNIVHSNGNTY (SEQ ID NO: 62)
VL-CDR2 for 1B2 antibody, defined by IMGT numbering system
KVS (SEQ ID NO: 63)
VL-CDR1 for 1B2 antibody, defined by Contact numbering system
VHSNGNTYLDWY (SEQ ID NO: 64)
VL-CDR2 for 1B2 antibody, defined by Contact numbering system
LLIYKVSNRF (SEQ ID NO: 65)
VL-CDR3 for 1B2 antibody, defined by Contact numbering system
FQGSHVPP (SEQ ID NO: 66)
Human CD40, uniprot #P25942-1
MVRLPLQCVLWGCLLTAVHPEPPTACREKQYLINSQCCSLCQPGQKLVSDCTEFTET
ECLPCGESEFLDTWNRETHCHQHKYCDPNLGLRVQQKGTSETDTICTCEEGWHCTS
EACESCVLHRSCSPGFGVKQIATGVSDTICEPCPVGFFSNVSSAFEKCHPWTSCETKD
LVVQQAGTNKTDVVCGPQDRLRALVVIPIIFGILFAILLVLVFIKKVAKKPTNKAPHP
KQEPQEINFPDDLPGSNTAAPVQETLHGCQPVTQEDGKESRISVQERQ (SEQ ID NO:
67)

1. An isolated monoclonal antibody, or an antigen-binding portion thereof, that specifically binds to CD40, comprising a heavy chain variable region comprising a CDR1 region, a CDR2 region and a CDR3 region, wherein the CDR1 region, the CDR2 region and the CDR3 region comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 1, 2 and 3, respectively; or (2) SEQ ID NOs: 37, 38 and 3, respectively; or (3) SEQ ID NOs: 39, 40 and 41, respectively; or (4) SEQ ID NOs: 42, 43 and 3, respectively; or (5) SEQ ID NOs: 44, 45 and 46, respectively; or (6) SEQ ID NOs: 4, 5 and 6, respectively; or (7) SEQ ID NOs: 47, 48 and 6, respectively; or (8) SEQ ID NOs: 49, 50 and 51, respectively; or (9) SEQ ID NOs: 52, 53 and 6, respectively; or (10) SEQ ID NOs: 54, 55 and 56, respectively.

2. The isolated monoclonal antibody, or the antigen-binding portion thereof, of claim 1, comprising a heavy chain variable region, wherein the heavy chain variable region comprises an amino acid sequence having at least 85%, 86%, 87%, 88%, 89%, 90%, 91%, 92%, 93%, 94%, 95%, 96%, 97%, 98%, 99% or 100% identity to SEQ ID NOs: 13, 14 (X1=C), 15 (X1=F), or 16.

3. The isolated monoclonal antibody, or the antigen-binding portion thereof, of claim 1, comprising a heavy chain constant region having an amino acid sequence with at least 85%, 86%, 87%, 88%, 89%, 90%, 91%, 92%, 93%, 94%, 95%, 96%, 97%, 98%, 99% or 100% identity to SEQ ID NO: 24, linked to the heavy chain variable region.

4. The isolated monoclonal antibody, or the antigen-binding portion thereof, of claim 1, comprising a heavy chain, wherein the heavy chain comprises an amino acid sequence having at least 85%, 86%, 87%, 88%, 89%, 90%, 91%, 92%, 93%, 94%, 95%, 96%, 97%, 98%, 99% or 100% identity to (1) SEQ ID NO: 26; or (2) SEQ ID NO: 27 (X1=C); or (3) SEQ ID NO: 28 (X1=F); or (4) SEQ ID NO: 29, respectively.

5. The isolated monoclonal antibody, or the antigen-binding portion thereof, of claim 1, comprising a light chain variable region comprising a CDR1 region, a CDR2 region and a CDR3 region, wherein the CDR1 region, the CDR2 region and the CDR3 region comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 7, 8 and 9, respectively; or (2) SEQ ID NOs: 57, 58 and 9, respectively; or (3) SEQ ID NOs: 59, 60 and 61, respectively; or (4) SEQ ID NOs: 10, 11 and 12, respectively; or (5) SEQ ID NOs: 62, 63 and 12, respectively; or (6) SEQ ID NOs: 64, 65 and 66, respectively.

6. The isolated monoclonal antibody, or the antigen-binding portion thereof, of claim 5, comprising a light chain variable region, wherein the light chain variable region comprises an amino acid sequence having at least 85%, 86%, 87%, 88%, 89%, 90%, 91%, 92%, 93%, 94%, 95%, 96%, 97%, 98%, 99% or 100% identity to SEQ ID NOs: 17, 18 (X1=S, X2=N, X3=F), 19 (X1=A, X2=N, X3=F), 20 (X1=S, X2=Y, X3=F), 21 (X1=S, X2=N, X3=Y), 22 (X1=A, X2=Y, X3=Y), or 23 (X1=M, X2=N, X3=A).

7. The isolated monoclonal antibody, or the antigen-binding portion thereof, of claim 5, comprising a light chain constant region having an amino acid sequence with at least 85%, 86%, 87%, 88%, 89%, 90%, 91%, 92%, 93%, 94%, 95%, 96%, 97%, 98%, 99% or 100% identity to SEQ ID NO: 25, linked to the light chain variable region.

8. The isolated monoclonal antibody, or the antigen-binding portion thereof, of claim 5, comprising a light chain, wherein the light chain comprises an amino acid sequence having at least 85%, 86%, 87%, 88%, 89%, 90%, 91%, 92%, 93%, 94%, 95%, 96%, 97%, 98%, 99% or 100% identity to (1) SEQ ID NO: 30; or (2) SEQ ID NO: 31 (X1=S, X2=N, X3=F); or (3) SEQ ID NO: 32 (X1=A, X2=N, X3=F); or (4) SEQ ID NO: 33 (X1=S, X2=Y, X3=F); or (5) SEQ ID NO: 34 (X1=S, X2=N, X3=Y); or (6) SEQ ID NO: 35 (X1=A, X2=Y, X3=Y); or (7) SEQ ID NO: 36 (X1=M, X2=N, X3=A), respectively.

9. The isolated monoclonal antibody, or an antigen-binding portion thereof, of claim 1, comprising a heavy chain variable region and a light chain variable region each comprising a CDR1 region, a CDR2 region and a CDR3 region, wherein the heavy chain variable region CDR1, CDR2 and CDR3, and the light chain variable region CDR1, CDR2 and CDR3 comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 1, 2, 3, 7, 8 and 9, respectively; or (2) SEQ ID NOs: 37, 38, 3, 7, 8, and 9, respectively; or (3) SEQ ID NOs: 39, 40, 41, 57, 58, and 9, respectively; or (4) SEQ ID NOs: 42, 43, 3, 7, 8, and 9, respectively; or (5) SEQ ID NOs: 44, 45, 46, 59, 60, and 61, respectively; or (6) SEQ ID NOs: 4, 5, 6, 10, 11, and 12, respectively; or (7) SEQ ID NOs: 47, 48, 6, 10, 11, and 12, respectively; or (8) SEQ ID NOs: 49, 50, 51, 62, 63, and 12, respectively; or (9) SEQ ID NOs: 52, 53, 6, 10, 11, and 12, respectively; or (10) SEQ ID NOs: 54, 55, 56, 64, 65, and 66, respectively,

10. The isolated monoclonal antibody, or an antigen-binding portion thereof, of claim 1, comprising a heavy chain variable region and a light chain variable region, wherein the heavy chain variable region and the light chain variable region comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 13 and 17, respectively; or (2) SEQ ID NOs: 14 (X1=C) and 18 (X1=S, X2=N, X3=F), respectively; or (3) SEQ ID NOs: 14 (X1=C) and 19 (X1=A, X2=N, X3=F), respectively; or (3) SEQ ID NOs: 14 (X1=C) and 20 (X1=S, X2=Y, X3=F), respectively; or (4) SEQ ID NOs: 14 (X1=C) and 21 (X1=S, X2=N, X3=Y), respectively; or (5) SEQ ID NOs: 14 (X1=C) and 22 (X1=A, X2=Y, X3=Y), respectively; or (6) SEQ ID NOs: 15 (X1=F) and 18 (X1=S, X2=N, X3=F), respectively; or (7) SEQ ID NOs: 15 (X1=F) and 22 (X1=A, X2=Y, X3=Y), respectively; or (8) SEQ ID NOs: 16 and 23 (X1=M, X2=N, X3=A), respectively.

11. The isolated monoclonal antibody, or the antigen-binding portion thereof, of claim 1, comprises a heavy chain and a light chain, wherein the heavy chain and the light chain comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 26 and 30, respectively; or (2) SEQ ID NOs: 27 (X1=C) and 31 (X1=S, X2=N, X3=F), respectively; or (3) SEQ ID NOs: 27 (X1=C) and 32 (X1=A, X2=N, X3=F), respectively; or (4) SEQ ID NOs: 27 (X1=C) and 33 (X1=S, X2=Y, X3=F), respectively; or (5) SEQ ID NOs: 27 (X1=C) and 34 (X1=S, X2=N, X3=Y), respectively; or (6) SEQ ID NOs: 27 (X1=C) and 35 (X1=A, X2=Y, X3=Y), respectively; or (7) SEQ ID NOs: 28 (X1=F) and 31 (X1=S, X2=N, X3=F), respectively; or (8) SEQ ID NOs: 28 (X1=F) and 35 (X1=A, X2=Y, X3=Y), respectively; or (9) SEQ ID NOs: 29 and 36 (X1=M, X2=N, X3=A), respectively.

12. The isolated monoclonal antibody, or the antigen-binding portion thereof, of claim 1, which (a) binds human or monkey CD40; (b) blocks humanCD40-humanCD40L interaction; and (c) activates CD40 signaling.

13. The isolated monoclonal antibody, or the antigen-binding portion thereof, of claim 1, which is a mouse, human, chimeric or humanized antibody.

14. The isolated monoclonal antibody, or the antigen-binding portion thereof, of claim 1, which is an IgG1, IgG2 or IgG4 isotype.

15. A pharmaceutical composition comprising the isolated monoclonal antibody, or antigen-binding portion thereof, of claim 1, and a pharmaceutically acceptable carrier.

16. A method for treating a cancer disease in a subject, comprising administering to the subject a therapeutically effective amount of the isolated monoclonal antibody, or the antigen-binding portion thereof, of claim 1.

17. The method of claim 16, wherein the cancer disease is a solid or non-solid tumor.

18. The method of claim 16, wherein the cancer disease is selected from the group consisting of B cell lymphoma, chronic lymphocytic leukemia, multiple myeloma, melanoma, colon adenocarcinoma, pancreas cancer, colon cancer, gastric intestine cancer, prostate cancer, bladder cancer, kidney cancer, ovary cancer, cervix cancer, breast cancer, lung cancer, and nasopharynx cancer.

19. The method of claim 16, further comprising administering an immune checkpoint antibody, a costimulatory antibody, a chemotherapeutic agent, and/or a cytokine.

20. The method of claim 19, wherein the immune checkpoint antibody is selected from the group consisting of an anti-VISTA antibody, an anti-PD-1 antibody, an anti-PD-L1 antibody, and an anti-LAG-3 antibody; wherein the costimulatory antibody is an anti-CD137 antibody or an anti-GITR antibody; wherein the chemotherapeutic agent is epirubicin, oxaliplatin, and/or 5-fluorouracil; wherein the cytokine is GM-CSF and/or IL-4.


SEQUENCE LISTING (73 sequences)

SEQ ID NO: 1 - protein, 5 aa, Artificial Sequence
  VH-CDR1 for mouse, chimeric and humanized C1C1 antibodies, defined by kabat numbering system
NYGMS

SEQ ID NO: 2 - protein, 17 aa, Artificial Sequence
  VH-CDR2 for mouse, chimeric and humanized C1C1 antibodies, defined by kabat numbering system
SISSGGDDTYYPDNVKG

SEQ ID NO: 3 - protein, 8 aa, Artificial Sequence
  VH-CDR3 for mouse, chimeric and humanized C1C1 antibodies, defined by kabat, Chothia or AbM numbering system
AGGKAMDY

SEQ ID NO: 4 - protein, 5 aa, Artificial Sequence
  VH-CDR1 for 1B2 antibody, defined by kabat numbering system
DYVMH

SEQ ID NO: 5 - protein, 17 aa, Artificial Sequence
  VH-CDR2 for 1B2 antibody, defined by kabat numbering system
YINPYNDGTKYNEKFKG

SEQ ID NO: 6 - protein, 10 aa, Artificial Sequence
  VH-CDR3 for 1B2 antibody, defined by kabat, Chothia or AbM numbering system
GFLRESWFGY

SEQ ID NO: 7 - protein, 11 aa, Artificial Sequence
  VL -CDR1 for mouse, chimeric and humanized C1C1 antibodies, defined by kabat, Chothia or AbM numbering system
RASQSIRDNLH

SEQ ID NO: 8 - protein, 7 aa, Artificial Sequence
  VL-CDR2 for mouse, chimeric and humanized C1C1 antibodies, defined by kabat, Chothia or AbM numbering system
YASQSIS

SEQ ID NO: 9 - protein, 9 aa, Artificial Sequence
  VL -CDR3 for mouse, chimeric and humanized C1C1 antibodies, defined by kabat, Chothia, IMGT or AbM numbering system
QQFNSWPLT

SEQ ID NO: 10 - protein, 16 aa, Artificial Sequence
  VL-CDR1 for 1B2 antibodies, defined by kabat, Chothia or AbM numbering system
RSSQNIVHSNGNTYLD

SEQ ID NO: 11 - protein, 7 aa, Artificial Sequence
  VL-CDR2 for 1B2 antibodies, defined by kabat, Chothia or AbM numbering system
KVSNRFS

SEQ ID NO: 12 - protein, 9 aa, Artificial Sequence
  VL-CDR3 for 1B2 antibodies, defined by kabat, Chothia, IMGT or AbM numbering system
FQGSHVPPT

SEQ ID NO: 13 - protein, 117 aa, Artificial Sequence
  VH for mouse and chimeric C1C1 antibodies
EVKLVESGGGLVKPGASLKLSCAASGFTFSNYGMSWVRQNSDKRLEWVASISSGGDDTYYPDNVKGRCTISRENAKNTLYLEMSSLKTEDTALYYCTRAGGKAMDYWGQGTSVTVST

SEQ ID NO: 14 - protein, 117 aa, Artificial Sequence
  VH for huC1C1-V1, huC1C1-V3, huC1C1-V4, huC1C1-V5 and huC1C1-V6
EVQLVESGGGVVQPGRSLRLSCAASGFTFSNYGMSWVRQAPGKGLEWVASISSGGDDTYYPDNVKGRCTISRDNSKNTLYLQMNSLRAEDTAVYYCTRAGGKAMDYWGQGTLVTVSS

SEQ ID NO: 15 - protein, 117 aa, Artificial Sequence
  VH for huC1C1-V2 and huC1C1-V7
EVQLVESGGGVVQPGRSLRLSCAASGFTFSNYGMSWVRQAPGKGLEWVASISSGGDDTYYPDNVKGRFTISRDNSKNTLYLQMNSLRAEDTAVYYCTRAGGKAMDYWGQGTLVTVSS

SEQ ID NO: 16 - protein, 119 aa, Artificial Sequence
  VH for 1B2
EVQLQQSGPELVKPGASVKMSCKASGYTFTDYVMHWVKQKPGQGLECIGYINPYNDGTKYNEKFKGKATLTSDKSSSAAYLELSSLTSEDSAVYYCARGFLRESWFGYWGQGTLVTVSA

SEQ ID NO: 17 - protein, 107 aa, Artificial Sequence
  VL for mouse and chimeric C1C1 antibodies
DIVLTQSPATLSVIPGNSVSLSCRASQSIRDNLHWYQQKSHESPRLLINYASQSISGIPSRFSGSGSGTDFTLTINSVETEDFGIYFCQQFNSWPLTFGGGTKLELK

SEQ ID NO: 18 - protein, 107 aa, Artificial Sequence
  VL for huC1C1-V1 and huC1C1-V2
EIVLTQSPATLSLSPGERATLSCRASQSIRDNLHWYQQKPGQSPRLLINYASQSISGIPARFSGSGSGTDFTLTISSLEPEDFAVYFCQQFNSWPLTFGQGTKVEIK

SEQ ID NO: 19 - protein, 107 aa, Artificial Sequence
  VL for huC1C1-V3
EIVLTQSPATLSLSPGERATLSCRASQSIRDNLHWYQQKPGQAPRLLINYASQSISGIPARFSGSGSGTDFTLTISSLEPEDFAVYFCQQFNSWPLTFGQGTKVEIK

SEQ ID NO: 20 - protein, 107 aa, Artificial Sequence
  VL for huC1C1-V4
EIVLTQSPATLSLSPGERATLSCRASQSIRDNLHWYQQKPGQSPRLLIYYASQSISGIPARFSGSGSGTDFTLTISSLEPEDFAVYFCQQFNSWPLTFGQGTKVEIK

SEQ ID NO: 21 - protein, 107 aa, Artificial Sequence
  VL for huC1C1-V5
EIVLTQSPATLSLSPGERATLSCRASQSIRDNLHWYQQKPGQSPRLLINYASQSISGIPARFSGSGSGTDFTLTISSLEPEDFAVYYCQQFNSWPLTFGQGTKVEIK

SEQ ID NO: 22 - protein, 107 aa, Artificial Sequence
  VL for huC1C1-V6 and huC1C1-V7
EIVLTQSPATLSLSPGERATLSCRASQSIRDNLHWYQQKPGQAPRLLIYYASQSISGIPARFSGSGSGTDFTLTISSLEPEDFAVYYCQQFNSWPLTFGQGTKVEIK

SEQ ID NO: 23 - protein, 112 aa, Artificial Sequence
  VL for 1B2
DVLMTQTPLSLPVSLGDQASISCRSSQNIVHSNGNTYLDWYLQKPGQSPKLLIYKVSNRFSGVPDRFSGSGSGTDFTLKINRVEAEDLGVYYCFQGSHVPPTFGAGTKLELK

SEQ ID NO: 24 - protein, 330 aa, Artificial Sequence
  CH, heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 107 aa, Artificial Sequence [sequence omitted]

SEQ ID NO: 26 - protein, 447 aa, Artificial Sequence
  Heavy chain for mouse and chimeric C1C1 antibodies
EVKLVESGGGLVKPGASLKLSCAASGFTFSNYGMSWVRQNSDKRLEWVASISSGGDDTYYPDNVKGRCTISRENAKNTLYLEMSSLKTEDTALYYCTRAGGKAMDYWGQGTSVTVSTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 447 aa, Artificial Sequence
  Heavy chain for huC1C1-V1, huC1C1-V3, huC1C1-V4, huC1C1-V5 and huC1C1-V6
EVQLVESGGGVVQPGRSLRLSCAASGFTFSNYGMSWVRQAPGKGLEWVASISSGGDDTYYPDNVKGRCTISRDNSKNTLYLQMNSLRAEDTAVYYCTRAGGKAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 447 aa, Artificial Sequence
  Heavy chain for huC1C1-V2 and huC1C1-V7
EVQLVESGGGVVQPGRSLRLSCAASGFTFSNYGMSWVRQAPGKGLEWVASISSGGDDTYYPDNVKGRFTISRDNSKNTLYLQMNSLRAEDTAVYYCTRAGGKAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 449 aa, Artificial Sequence
  Heavy chain for 1B2
EVQLQQSGPELVKPGASVKMSCKASGYTFTDYVMHWVKQKPGQGLECIGYINPYNDGTKYNEKFKGKATLTSDKSSSAAYLELSSLTSEDSAVYYCARGFLRESWFGYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 214 aa, Artificial Sequence
  Light chain for mouse and chimeric C1C1 antibodies
DIVLTQSPATLSVIPGNSVSLSCRASQSIRDNLHWYQQKSHESPRLLINYASQSISGIPSRFSGSGSGTDFTLTINSVETEDFGIYFCQQFNSWPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 214 aa, Artificial Sequence
  Light chain for huC1C1-V1 and huC1C1-V2
EIVLTQSPATLSLSPGERATLSCRASQSIRDNLHWYQQKPGQSPRLLINYASQSISGIPARFSGSGSGTDFTLTISSLEPEDFAVYFCQQFNSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 214 aa, Artificial Sequence
  Light chain for huC1C1-V3
EIVLTQSPATLSLSPGERATLSCRASQSIRDNLHWYQQKPGQAPRLLINYASQSISGIPARFSGSGSGTDFTLTISSLEPEDFAVYFCQQFNSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 214 aa, Artificial Sequence
  Light chain for huC1C1-V4
EIVLTQSPATLSLSPGERATLSCRASQSIRDNLHWYQQKPGQSPRLLIYYASQSISGIPARFSGSGSGTDFTLTISSLEPEDFAVYFCQQFNSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 214 aa, Artificial Sequence
  Light chain for huC1C1-V5
EIVLTQSPATLSLSPGERATLSCRASQSIRDNLHWYQQKPGQSPRLLINYASQSISGIPARFSGSGSGTDFTLTISSLEPEDFAVYYCQQFNSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 214 aa, Artificial Sequence
  Light chain for huC1C1-V6 and huC1C1-V7
EIVLTQSPATLSLSPGERATLSCRASQSIRDNLHWYQQKPGQAPRLLIYYASQSISGIPARFSGSGSGTDFTLTISSLEPEDFAVYYCQQFNSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 219 aa, Artificial Sequence
  Light chain for 1B2
DVLMTQTPLSLPVSLGDQASISCRSSQNIVHSNGNTYLDWYLQKPGQSPKLLIYKVSNRFSGVPDRFSGSGSGTDFTLKINRVEAEDLGVYYCFQGSHVPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 7 aa, Artificial Sequence
  VH-CDR1 for mouse, chimeric and humanized C1C1 antibodies, defined by Chothia numbering system
GFTFSNY

SEQ ID NO: 38 - protein, 6 aa, Artificial Sequence
  VH-CDR2 for mouse, chimeric and humanized C1C1 antibodies, defined by Chothia numbering system
SSGGDD

SEQ ID NO: 39 - protein, 8 aa, Artificial Sequence
  VH-CDR1 for mouse, chimeric and humanized C1C1 antibodies, defined by IMGT numbering system
GFTFSNYG

SEQ ID NO: 40 - protein, 7 aa, Artificial Sequence
  VH-CDR2 for mouse, chimeric and humanized C1C1 antibodies, defined by IMGT numbering system
ISSGGDD

SEQ ID NO: 41 - protein, 10 aa, Artificial Sequence
  VH-CDR3 for mouse, chimeric and humanized C1C1 antibodies, defined by IMGT numbering system
TRAGGKAMDY

SEQ ID NO: 42 - protein, 10 aa, Artificial Sequence
  VH-CDR1 for mouse, chimeric and humanized C1C1 antibodies, defined by AbM numbering system
GFTFSNYGMS

SEQ ID NO: 43 - protein, 10 aa, Artificial Sequence
  VH-CDR2 for mouse, chimeric and humanized C1C1 antibodies, defined by AbM numbering system
SISSGGDDTY

SEQ ID NO: 44 - protein, 6 aa, Artificial Sequence
  VH-CDR1 for mouse, chimeric and humanized C1C1 antibodies, defined by Contact numbering system
SNYGMS

SEQ ID NO: 45 - protein, 13 aa, Artificial Sequence
  VH-CDR2 for mouse, chimeric and humanized C1C1 antibodies, defined by Contact numbering system
WVASISSGGDDTY

SEQ ID NO: 46 - protein, 9 aa, Artificial Sequence
  VH-CDR3 for mouse, chimeric and humanized C1C1 antibodies, defined by Contact numbering system
TRAGGKAMD

SEQ ID NO: 47 - protein, 7 aa, Artificial Sequence
  VH-CDR1 for 1B2 antibody, defined by Chothia numbering system
GYTFTDY

SEQ ID NO: 48 - protein, 6 aa, Artificial Sequence
  VH-CDR2 for 1B2 antibody, defined by Chothia numbering system
NPYNDG

SEQ ID NO: 49 - protein, 8 aa, Artificial Sequence
  VH-CDR1 for 1B2 antibody, defined by IMGT numbering system
GYTFTDYV

SEQ ID NO: 50 - protein, 8 aa, Artificial Sequence
  VH-CDR2 for 1B2 antibody, defined by IMGT numbering system
INPYNDGT

SEQ ID NO: 51 - protein, 12 aa, Artificial Sequence
  VH-CDR3 for 1B2 antibody, defined by IMGT numbering system
ARGFLRESWFGY

SEQ ID NO: 52 - protein, 10 aa, Artificial Sequence
  VH-CDR1 for 1B2 antibody, defined by AbM numbering system
GYTFTDYVMH

SEQ ID NO: 53 - protein, 10 aa, Artificial Sequence
  VH-CDR2 for 1B2 antibody, defined by AbM numbering system
YINPYNDGTK

SEQ ID NO: 54 - protein, 6 aa, Artificial Sequence
  VH-CDR1 for 1B2 antibody, defined by Contact numbering system
TDYVMH

SEQ ID NO: 55 - protein, 13 aa, Artificial Sequence
  VH-CDR2 for 1B2 antibody, defined by Contact numbering system
CIGYINPYNDGTK

SEQ ID NO: 56 - protein, 11 aa, Artificial Sequence
  VH-CDR3 for 1B2 antibody, defined by Contact numbering system
ARGFLRESWFG

SEQ ID NO: 57 - protein, 6 aa, Artificial Sequence
  VL -CDR1 for mouse, chimeric and humanized C1C1 antibodies, defined by IMGT numbering system
QSIRDN

SEQ ID NO: 58 - protein, 3 aa, Artificial Sequence
  VL-CDR2 for mouse, chimeric and humanized C1C1 antibodies, defined by IMGT numbering system
YAS

SEQ ID NO: 59 - protein, 7 aa, Artificial Sequence
  VL -CDR1 for mouse, chimeric and humanized C1C1 antibodies, defined by Contact numbering system
RDNLHWY

SEQ ID NO: 60 - protein, 10 aa, Artificial Sequence
  VL-CDR2 for mouse, chimeric and humanized C1C1 antibodies, defined by Contact numbering system
LLINYASQSI

SEQ ID NO: 61 - protein, 8 aa, Artificial Sequence
  VL -CDR3 for mouse, chimeric and humanized C1C1 antibodies, defined by Contact numbering system
QQFNSWPL

SEQ ID NO: 62 - protein, 11 aa, Artificial Sequence
  VL-CDR1 for 1B2 antibody, defined by IMGT numbering system
QNIVHSNGNTY

SEQ ID NO: 63 - protein, 3 aa, Artificial Sequence
  VL-CDR2 for 1B2 antibody, defined by IMGT numbering system
KVS

SEQ ID NO: 64 - protein, 12 aa, Artificial Sequence
  VL-CDR1 for 1B2 antibody, defined by Contact numbering system
VHSNGNTYLDWY

SEQ ID NO: 65 - protein, 10 aa, Artificial Sequence
  VL-CDR2 for 1B2 antibody, defined by Contact numbering system
LLIYKVSNRF

SEQ ID NO: 66 - protein, 8 aa, Artificial Sequence
  VL-CDR3 for 1B2 antibody, defined by Contact numbering system
FQGSHVPP

SEQ ID NO: 67 - protein, 277 aa, Homo sapiens
  Human CD40, uniprot #P25942-1
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 68 - DNA, 351 bp, Artificial Sequence
  Nucleotides encoding VH for mouse and chimeric C1C1 antibodies
gaagtgaagctggtggagtctgggggaggcttagtgaagcctggagcgtctctgaaactctcctgtgcagcctctggattcactttcagtaactatggcatgtcttgggttcgccagaattcagacaagaggctggagtgggtcgcatccattagtagtggtggtgatgacacctactatccagacaatgtaaagggccgatgcaccatctccagagagaatgccaagaacaccctgtatttggagatgagtagtctgaagactgaggacacggccttgtattactgtacaagagctggggggaaggctatggactactggggtcaaggaacctcagtcaccgtctccaca

SEQ ID NO: 69 - DNA, 357 bp, Artificial Sequence
  Nucleotides encoding VH for 1B2
gaggtccagctgcagcagtctggacctgagctggtaaagcctggggcttcagtgaagatgtcctgcaaggcttctggatacacattcactgactatgttatgcactgggtgaagcagaagcctgggcagggccttgagtgtattggatatattaatccttacaatgatggtactaagtacaatgagaagttcaaaggcaaggccacactgacttcagacaaatcctccagcgcagcctacctggagctcagcagcctgacctctgaggactctgcggtctattactgtgcaagagggttcctacgagagtcctggtttggttactggggccaagggactctggtcactgtctctgca

SEQ ID NO: 70 - DNA, 321 bp, Artificial Sequence
  Nucleotides encoding VL for mouse and chimeric C1C1 antibodies
gacattgtgctaactcagtctccagccaccctgtctgtgattccaggaaatagcgtcagtctttcctgcagggccagccaaagtattcgcgacaacctacactggtatcaacaaaagtcacatgagtctccaaggcttctcatcaactatgcttcccagtccatctctgggatcccctcccggttcagtggcagtggatcagggacagatttcactctcactatcaacagtgtggagactgaagattttggaatatatttctgtcagcaatttaacagctggcccctcacgttcggtggtgggaccaagctggagctgaaa

SEQ ID NO: 71 - DNA, 336 bp, Artificial Sequence
  Nucleotides encoding VL for 1B2
gatgttttgatgacccaaactccactctccctgcctgtcagtcttggagatcaagcctccatctcttgcagatctagtcagaacattgtacatagtaatggaaacacctatttagattggtacctgcagaaaccaggccagtctccaaagctcctgatctacaaagtttccaaccgattttctggggtcccagacaggttcagtggcagtggatcagggacagatttcacactcaagatcaacagagtggaggctgaggatctgggagtttattactgctttcaaggttcacatgttcctcctacgttcggtgctgggaccaagctggagctgaaa

SEQ ID NO: 72 - DNA, 990 bp, Artificial Sequence
  Nucleotides encoding CH
gcttctactaaaggtccttccgtttttcccttagccccatcatcgaagagtaccagcggcggaacagcagcgttggggtgtcttgtcaaagattatttcccggaacctgtaacggtgtcttggaattccggtgctctcacttcaggcgttcatacctttcccgccgtcctacaatcgagtggactgtacagcttatcttccgtagtgacagttccatcatcgagtttggggacgcagacttatatttgcaacgtcaatcacaagccgagcaacaccaaagtagacaagaaagtggagcctaagtcttgtgataaaacacatacgtgccccccatgtccggcacctgaacttctcggtggcccctccgttttcctatttccaccgaagcctaaagacactctgatgatctcacgtacccccgaggtcacatgcgtagtggttgatgtctcgcacgaagacccagaggtaaagttcaattggtacgtggatggagttgaagtccataacgcgaaaacgaagccgcgcgaggaacaatataatagtacttaccgagtagtgagcgttttaaccgtcttgcaccaggactggcttaacgggaaagagtataagtgtaaagtatctaataaggctctccctgcccccatagaaaaaacaatttccaaggcaaaaggtcaaccacgggagccgcaggtgtacacgctacctccctcaagagaagagatgactaagaaccaagtttcgctgacctgcttagtcaaaggcttttatccaagtgatatcgcggtagaatgggagagcaatggacagccggaaaacaattacaagacaacgcctcccgtgttggactctgatgggtccttctttctttattcaaaactcactgttgacaagtcgaggtggcaacagggtaacgtcttcagttgtagcgtaatgcatgaggctctacacaatcattacacccaaaaatctctgtccttatcaccaggcaag

SEQ ID NO: 73 - DNA, 324 bp, Artificial Sequence
  Nucleotides encoding CL
cgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga